(19) United States
(12) Patent Application Publication
(10) Pub. No.: US 2026/0098020 A1
(43) Pub. Date: Apr. 9, 2026

(54) HERBICIDAL PYRIMIDINYLMETHYL-SUBSTITUTED 1,2,4-TRIAZOLES

(71) Applicant: FMC CORPORATION, Philadelphia, PA (US)

(72) Inventor: Paula Louise SHARPE, Middletown, DE (US)

(21) Appl. No.: 19/348,862

(22) Filed: Oct. 3, 2025

Related U.S. Application Data
(60) Provisional application No. 63/702,655, filed on Oct. 3, 2024.

(51) Int. Cl. C07D 249/08; A01N 43/653; A01N 43/76; A01P 13/00; C07D 263/36; C07D 333/04

(52) U.S. Cl. CPC C07D 249/08; A01N 43/653; A01N 43/76; A01P 13/00; C07D 263/36; C07D 333/04

(57) ABSTRACT

Disclosed are a compound of Formula 1, stereoisomers, N-oxides, and salts thereof, agricultural compositions containing them and their use as herbicides:

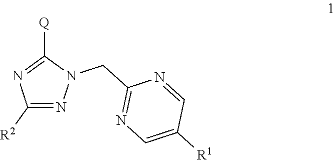

wherein R¹, R², Q and R³ are as defined in the disclosure of the invention.

Also disclosed are compositions containing the compounds of Formula 1 and methods for controlling undesired vegetation comprising contacting the undesired vegetation or its environment with an effective amount of a compound or a composition of the invention.

### FIELD OF THE INVENTION

This invention relates to certain compound of Formula 1, stereoisomers, N-oxides, and salts thereof; their compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action. WO 2022/013293, WO 2023/099354, WO 2023/066783 and WO 2024/074414 disclose certain compounds and their use as herbicides. The compounds of the present invention are not disclosed in these publications.

### SUMMARY OF THE INVENTION

This invention is directed to a compound of Formula 1, stereoisomers, N-oxides, and salts thereof, agricultural compositions containing them and their use as herbicides:

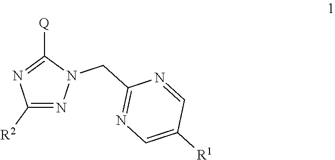

wherein
R¹ is halogen, cyano, nitro, C₁-C₄ alkyl, C₁-C₄ haloalkyl, C₃-C₇ cycloalkyl, C₂-C₅ alkenyl, C₂-C₅ alkynyl, C₁-C₄ alkylthio, C₁-C₄ alkylsulfinyl, C₁-C₄ alkylsulfonyl, C₁-C₄ alkoxy, C₂-C₅ alkylcarbonyl, C₂-C₅ alkoxycarbonyl, C₁-C₄ haloalkoxy or C₂-C₇ alkoxyalkyl;
R² is cyano, nitro, halogen, C₁-C₄ alkyl, C₁-C₄ haloalkyl, C₁-C₄ haloalkoxy, C₂-C₇ alkoxyalkyl, C₂-C₇ alkoxyalkoxy, C₃-C₇ cycloalkyl, C₂-C₅ alkylcarbonyl, C₂-C₅ alkoxycarbonyl, C₁-C₄ alkylthio, C₁-C₄ alkylsulfinyl, C₁-C₄ alkylsulfonyl, C₁-C₄ haloalkylthio, C₁-C₄ haloalkylsulfinyl or C₁-C₄ haloalkylsulfonyl;
Q is C₁-C₆ alkyl, C₁-C₆ haloalkyl, C₃-C₆ cycloalkyl, C₁-C₆ alkylthio, C₁-C₆ haloalkylthio, C₁-C₆ alkylsulfinyl, C₁-C₆ alkylsulfonyl, C₁-C₆ alkoxy, C₁-C₆ haloalkoxy, C₂-C₅ alkylcarbonyl, C₂-C₅ alkoxycarbonyl, C₂-C₅ haloalkylcarbonyl, C₂-C₅ haloalkoxycarbonyl or C₁-C₄ alkoxyalkyl; or G;
G is phenyl, or a 5- or 6-membered heterocyclic ring; the phenyl or heterocyclic ring unsubstituted or substituted with 1 or 2 R³; and
R³ is halogen, cyano, nitro, C₁-C₄ alkyl, C₁-C₄ haloalkyl, C₃-C₅ cycloalkyl, C₁-C₄ alkenyl, C₁-C₄ alkynyl, C₁-C₄ alkylthio, C₁-C₄ alkylsulfinyl, C₁-C₄ alkylsulfonyl, C₁-C₄ alkoxy, C₁-C₄ haloalkoxy, C₂-C₅ alkylcarbonyl, C₂-C₅ alkoxycarbonyl, C₁-C₄ alkoxyalkyl, C₁-C₄ alkylthio, C₁-C₄ alkylsulfinyl or C₁-C₄ alkylsulfonyl.

This invention relates to a compound of Formula 1 as defined and described in the Summary of the Invention. This invention also relates to composition comprising a compound of Formula 1 as defined and described in the Summary of the Invention. This invention also relates to a method for controlling unwanted vegetation comprising applying a herbicidally effective amount of a compound of Formula 1 or a composition comprising a compound of Formula 1 as defined and described above in Summary of the Invention. This invention further relates to a method for controlling the growth of undesired vegetation comprising contacting the vegetation or its environment with a herbicidally effective amount of a compound of Formula 1 as described in the Summary of the Invention. This invention also includes a herbicidal mixture comprising (a) a compound selected from Formula 1, N-oxides, and salts thereof, and (b) at least one additional active ingredient selected from (b1) through (b18); and salts of compounds of (b1) through (b18), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a compound, mixture, method or process that comprises a list of elements is not necessarily limited to only those elements but may include other elements not expressly listed or inherent to such compound, composition, mixture, method or process.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a compound, composition, mixture, method or process that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Cycloalkyl” includes, for example, cyclopropyl, cyclobutyl, cyclopentyl and cyclohexyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkylthio”, and the like, are is defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. “Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers.

The total number of carbon atoms in a substituent group is indicated by the “C₁-C₆” prefix where i and j are numbers from 1 to 6. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents, e.g., ([R^{v})ᵣ], r is 1, 2, 3, 4 or 5).

When one or more positions on a group are said to be “not substituted” or “unsubstituted”, then hydrogen atoms are attached to take up any free valency.

Unless otherwise indicated, a “ring” as a component of Formula 1 (e.g., substituent G) is heterocyclic. The term “ring member” refers to an atom or other moiety (e.g., C(═O), C(═S), S(O) or S(O)₂) forming the backbone of a ring or ring system.

The terms “heterocyclic ring” or “heterocycle” denote a ring or ring system in which at least one atom forming the ring backbone is not carbon, e.g., nitrogen, oxygen or sulfur.

Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen. “Aromatic” indicates that each of the ring atoms is essentially in the same plane and has a p-orbital perpendicular to the ring plane, and that (4n+2) π electrons, where n is a positive integer, are associated with the ring to comply with Hückel's rule.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “unsubstituted or substituted” or with the term “(un)substituted.” Unless otherwise indicated, an optionally substituted group may have a substituent at each substitutable position of the group, and each substitution is independent of the other.

When G is a 5- or 6-membered nitrogen-containing heterocyclic ring, it may be attached to the remainder of Formula 1 though any available carbon or nitrogen ring atom, unless otherwise described. As noted above, G can be (among others) phenyl substituted or unsubstituted with one or more substituents selected from a group of substituents as defined in the Summary of the Invention. An example of phenyl optionally substituted with one to five substituents is the ring illustrated as U-1 in Exhibit 1, wherein (R^{v})ᵣ is as defined in the Summary of the Invention for R³ and r is an integer (from 0 to 2).

As noted above, G can be (among others) 5- or 6-membered heterocyclic ring, which may be saturated or unsaturated, optionally substituted with one or more substituents selected from a group of substituents as defined in the Summary of the Invention. Examples of a 5- or 6-membered unsaturated aromatic heterocyclic ring optionally substituted with from one or more substituents include the rings U-2 through U-61 illustrated in Exhibit 1 wherein R^{v} is any substituent as defined in the Summary of the Invention for G (i.e. (R³)ᵣ) and r is an integer from 0 to 2, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

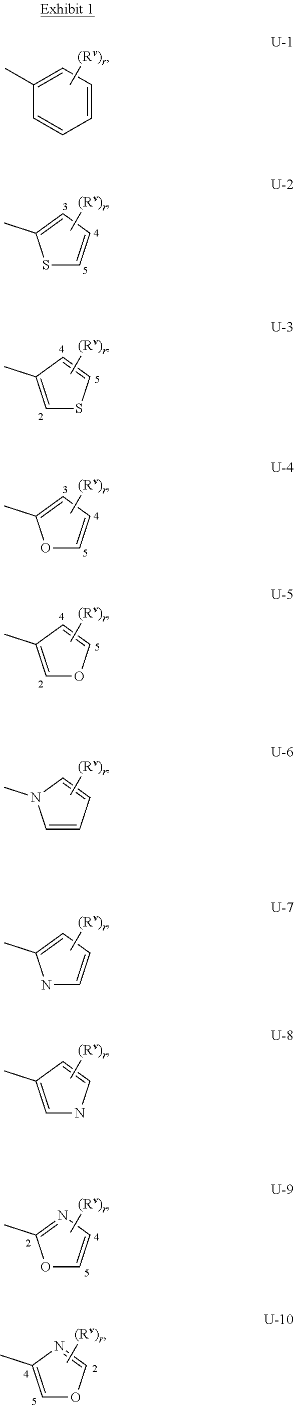

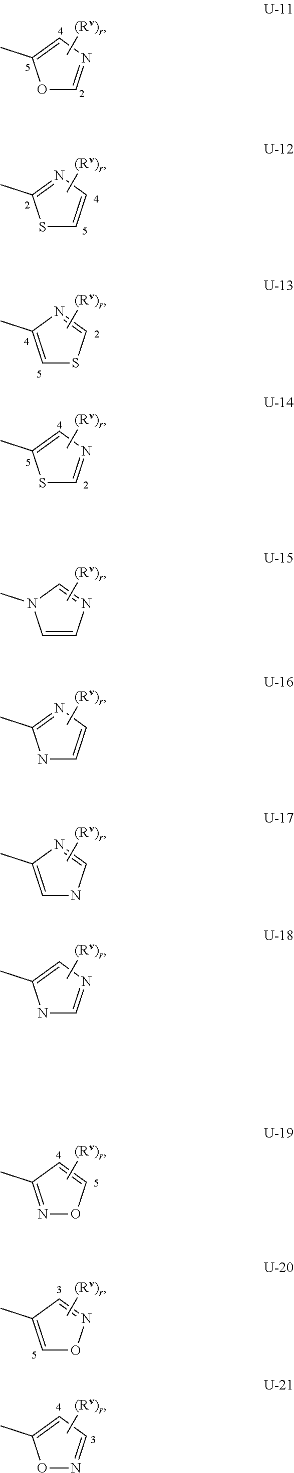

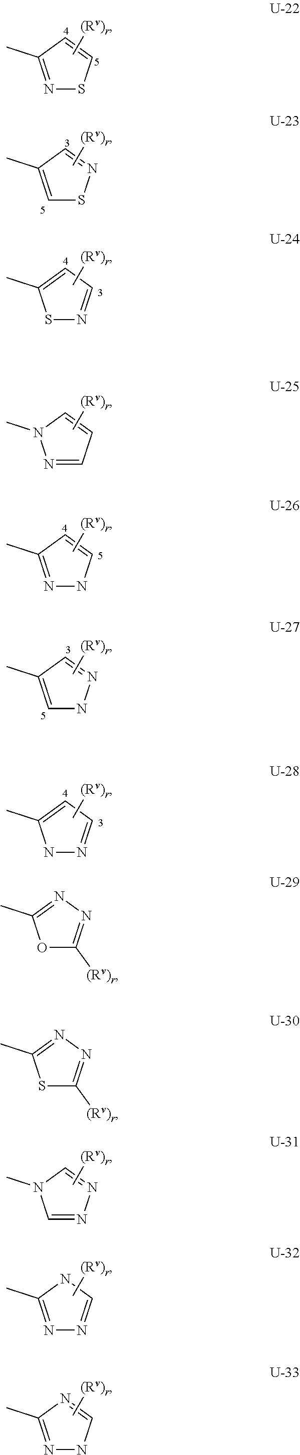

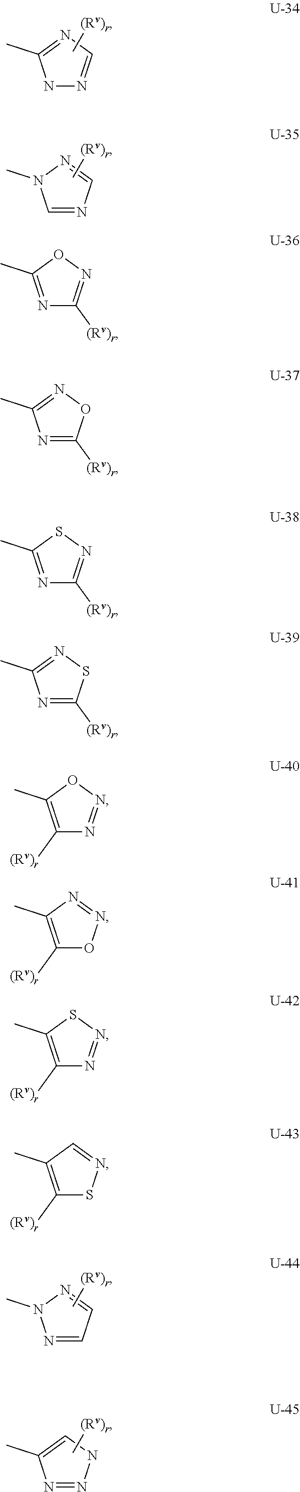

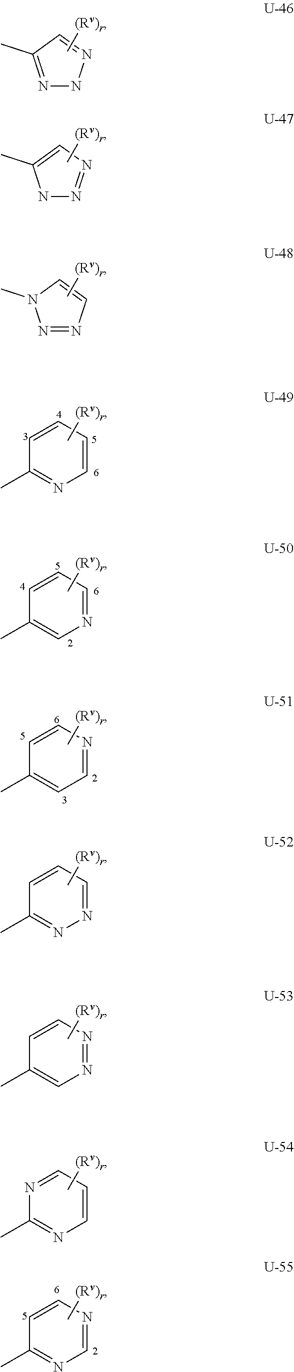

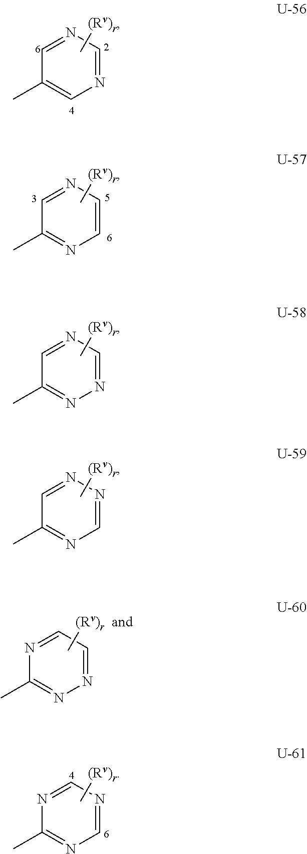

A wide variety of synthetic methods are known in the art to enable preparation of aromatic heterocyclic rings; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

This invention comprises racemic mixtures, for example, equal amounts of the two enantiomers. In addition, this invention includes compounds that are enriched compared to the racemic mixture in an enantiomer of Formula 1. Also included are the essentially pure enantiomers of compounds of Formula 1.

When enantiomerically enriched, one enantiomer is present in greater amounts than the other, and the extent of enrichment can be defined by an expression of enantiomeric excess (“ee”), which is defined as (2x-1)·100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can comprise additional chiral centers. For example, substituents and other molecular constituents such as R² and R³ may contain chiral centers. This invention comprises racemic mixtures as well as enriched and essentially pure stereoconfigurations at these additional chiral centers.

Compounds of this invention can exist as one or more conformational isomers due to restricted rotation about the bond (e.g., C—Q) in Formula 1. This invention comprises mixtures of conformational isomers. In addition, this invention includes compounds that are enriched in one conformer relative to others.

Compounds of Formula 1 typically exists in more than one form, and Formula 1 thus includes all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Invention include (i.e. where Formula 1 as used in the following Embodiments includes stereoisomers, N-oxides and salts thereof):
Embodiment 1. A compound of Formula 1, stereoisomers, N-oxides, and salts thereof, agricultural compositions containing them and their use as herbicides as described in the Summary of the Disclosure.
Embodiment 2. A compound of Formula 1 or Embodiment 1 wherein R¹ is halogen, nitro or C₁-C₃ alkyl.
Embodiment 3. A compound of Embodiment 2 wherein R¹ is halogen, nitro or C₁-C₂ alkyl.
Embodiment 4. A compound of Embodiment 3 wherein R¹ is F, Cl, Br, nitro or CH₃.
Embodiment 5. A compound of Embodiment 4 wherein R¹ is Cl, Br, nitro or CH₃.
Embodiment 6. A compound of Embodiment 5 wherein R¹ is Cl or Br.
Embodiment 7. A compound of Embodiment 6 wherein R¹ is Cl.
Embodiment 8. A compound of Formula 1 or any one of Embodiments 1 through 7 wherein R² is cyano, nitro, halogen, C₁-C₄ alkyl or C₁-C₄ haloalkyl.
Embodiment 9. A compound of Embodiment 8 wherein R² is C₁-C₄ alkyl or C₁-C₄ haloalkyl.
Embodiment 10. A compound of Embodiment 9 wherein R² is C₁-C₂ alkyl or C₁-C₂ haloalkyl.
Embodiment 11. A compound of Embodiment 10 wherein R² is C₁-C₂ haloalkyl.
Embodiment 12. A compound of Embodiment 11 wherein R² is CHF₂, CF₃ or CF₂Cl.
Embodiment 13. A compound of Embodiment 12 wherein R² is CHF₂ or CF₃.
Embodiment 14. A compound of Embodiment 13 wherein R² is CF₃.
Embodiment 15. A compound of Formula 1 or any one of Embodiments 1 through 14 wherein Q is C₁-C₆ alkyl, C₁-C₆ haloalkyl, C₁-C₆ alkylthio, C₁-C₆ haloalkylthio, C₁-C₆ alkylsulfinyl, C₁-C₆ alkylsulfonyl, C₁-C₆ alkoxy, C₁-C₆ haloalkoxy, C₂-C₅ alkylcarbonyl, C₂-C₅ alkoxycarbonyl, C₂-C₅ haloalkylcarbonyl, C₂-C₅ haloalkoxycarbonyl; or G;
Embodiment 16. A compound of Embodiment 15 wherein Q is C₁-C₆ alkyl, C₁-C₆ haloalkyl, C₁-C₆ haloalkylthio, C₁-C₆ haloalkoxy, C₂-C₅ alkylcarbonyl, C₂-C₅ alkoxycarbonyl or C₂-C₅ haloalkylcarbonyl; or G.
Embodiment 17. A compound of Embodiment 16 wherein Q is C₃-C₆ alkyl, C₃-C₆ haloalkyl, C₃-C₆ haloalkylthio, C₃-C₆ haloalkoxy, C₃-C₅ alkylcarbonyl, C₃-C₅ alkoxycarbonyl or C₃-C₅ haloalkylcarbonyl; or G.
Embodiment 18. A compound of Embodiment 16 wherein Q is C₁-C₆ alkyl, C₃-C₆ haloalkyl, C₃-C₆ haloalkylthio, C₃-C₆ haloalkoxy, C₃-C₅ alkylcarbonyl, C₃-C₅ alkoxycarbonyl or C₃-C₅ haloalkylcarbonyl.
Embodiment 19. A compound of Embodiment 16 wherein Q is C₁-C₆ alkyl or C₃-C₆ haloalkyl; or G.
Embodiment 20. A compound of Embodiment 16 wherein Q is C₁-C₆ alkyl, C₃-C₆ haloalkyl.
Embodiment 21. A compound of Embodiment 16 wherein Q is —CH₂CF₃, —CH₂CH₂CF₃, —CH₂CH₂CH₂CF₃, —OCH₂CF₃, —OCH₂CH₂CF₃, —OCH₂CH₂CH₂CF₃, —C(═O)CH₂CH₂CH₃, —C(═O)CH₂CH₂CH₂CH₃, —C(═O)CH₂CH₂CF₃, —C(═O)CH₂CH₂CH₂CF₃; or G.
Embodiment 22. A compound of Embodiment 16 wherein Q is —CH₂CF₃, —CH₂CH₂CF₃, —CH₂CH₂CH₂CF₃, —OCH₂CF₃, —OCH₂CH₂CF₃, —OCH₂CH₂CH₂CF₃, —C(═O)CH₂CH₂CH₃, —C(═O)CH₂CH₂CH₂CH₃, —C(═O)CH₂CH₂CF₃, —C(═O)CH₂CH₂CH₂CF₃.
Embodiment 23. A compound of Embodiment 19 wherein Q is —CH₂CF₃, —CH₂CH₂CF₃—CH₂CH₂CH₂CF₃, or —OCH₂CH₂CH₂CF₃; or G.
Embodiment 24. A compound of Embodiment 20 wherein Q is —CH₂CF₃, —CH₂CH₂CF₃—CH₂CH₂CH₂CF₃, or —OCH₂CH₂CH₂CF₃.
Embodiment 25. A compound of Embodiment 21 wherein Q is —CH₂CF₃, —CH₂CH₂CF₃, or —CH₂CH₂CH₂CF₃; or G.
Embodiment 26. A compound of Embodiment 22 wherein Q is —CH₂CF₃, —CH₂CH₂CF₃ or —CH₂CH₂CH₂CF₃.
Embodiment 27. A compound of Formula 1 or any one of Embodiments 1 through 17, 19, 21, 23 or 25 wherein G is phenyl substituted with 1 or 2 R³; or a 5- or 6-membered heterocyclic ring substituted with 1 or 2 R³.
Embodiment 28. A compound of Embodiment 27 wherein
   R³ is R^{3a}, R^{3b}, R^{3c}, R^{3d} or R^{3e}; and
   G is selected from

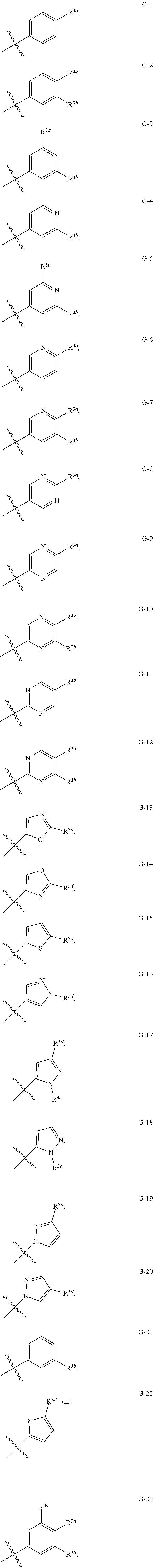

Embodiment 29. A compound of Embodiment 28 wherein G is G-1.
Embodiment 30. A compound of Embodiment 29 wherein G is selected from G-4, G-5, G-6 and G-7.
Embodiment 31. A compound of Embodiment 30 wherein G is selected from G-4 and G-6.
Embodiment 32. A compound of Embodiment 31 wherein G is G-4.
Embodiment 33. A compound of Embodiment 31 wherein G is G-6.
Embodiment 34. A compound of Embodiment 28 wherein G is selected from G-8, G-9, G-10, G-11 and G-12.
Embodiment 35. A compound of Embodiment 34 wherein G is selected from G-8, G-9 and G-11.
Embodiment 36. A compound of Embodiment 35 wherein G is selected from G-8.
Embodiment 37. A compound of Embodiment 35 wherein G is selected from G-11.
Embodiment 38. A compound of Embodiment 28 wherein G is selected from G-13, G-14, G-15, G-16, G-17 and G-18.
Embodiment 39. A compound of Embodiment 38 wherein G is selected from G-13, G-14 and G-15.
Embodiment 40. A compound of Embodiment 39 wherein G is selected from G-13 or G-14.
Embodiment 41. A compound of Embodiment 40 wherein G is selected from G-13.
Embodiment 42. A compound of Embodiment 40 wherein G is selected from G-14.
Embodiment 43. A compound of Embodiment 28 wherein G is selected from G-1, G-2, G-4, G-5, G-6, G-7, G-8, G-15 and G-16.
Embodiment 44. A compound of Embodiment 43 wherein G is selected from G-1, G-2, G-6, G-7, G-8 and G-15.
Embodiment 45. A compound of Embodiment 44 wherein G is G-1, G-2, G-6 and G-7.
Embodiment 46. A compound of Embodiment 44 wherein G is G-1 and G-2.
Embodiment 47. A compound of Embodiment 45 wherein G is G-2.
Embodiment 48. A compound of Embodiment 45 wherein G is G-7.
Embodiment 49. A compound any of Embodiments 1 through 17, 19, 21, 23 or 25 wherein Q is G (i.e. Q is other than C₁-C₆ alkyl, C₁-C₆ haloalkyl, C₃-C₆ cycloalkyl, C₁-C₆ alkylthio, C₁-C₆ haloalkylthio, C₁-C₆ alkylsulfinyl, C₁-C₆ alkylsulfonyl, C₁-C₆ alkoxy, C₁-C₆ haloalkoxy, C₂-C₅ alkylcarbonyl, C₂-C₅ alkoxycarbonyl, C₂-C₅ haloalkylcarbonyl, C₂-C₅ haloalkoxycarbonyl or C₁-C₄ alkoxyalkyl).
Embodiment 50. A compound of Formula 1 or any of Embodiments 1 through 48 wherein R³ is halogen, cyano, C₁-C₄ alkyl, C₁-C₄ haloalkyl, C₁-C₄ alkylsulfinyl, C₁-C₄ alkylsulfonyl, C₁-C₄ alkoxy or C₁-C₄ alkylthio.
Embodiment 51. A compound of Embodiment 50 wherein R³ is halogen, C₁-C₃ alkyl or C₁-C₃ haloalkyl.
Embodiment 52. A compound of Embodiment 51 wherein R³ is halogen, C₁-C₂ alkyl or C₁-C₂ haloalkyl.
Embodiment 53. A compound of Embodiment 53 wherein R³ is Cl, F, CHF₂, CH₃ or CF₃.
Embodiment 54. A compound of Embodiment 53 wherein R^{3a} is Cl, F or CF₃.
Embodiment 55. A compound of Embodiment 53 wherein R^{3a} is Cl or CF₃.
Embodiment 56. A compound of Embodiment 53 wherein R^{3a} is F.
Embodiment 57. A compound of Embodiment 53 wherein R^{3a} is CF₃.
Embodiment 58. A compound of Embodiment 53 wherein R^{3b} is Cl or F.
Embodiment 59. A compound of Embodiment 53 wherein R^{3b} is Cl.
Embodiment 60. A compound of Embodiment 53 wherein R^{3b} is F.
Embodiment 61. A compound of Embodiment 53 wherein R^{3c} is Cl.
Embodiment 62. A compound of Embodiment 53 wherein R^{3d} is Cl, CHF₂ or CF₃.
Embodiment 63. A compound of Embodiment 62 wherein R^{3d} is CHF₂.
Embodiment 64. A compound of Embodiment 52 wherein R^{3e} is Cl.

Embodiments of this disclosure, including Embodiments 1-64 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this disclosure, including Embodiments 1-64 above as well as any other embodiments described herein, and any combination thereof, pertain to the compositions comprising a compound of Formula 1 and methods of using the compound of Formula 1 of the present disclosure.

Combinations of Embodiments 1-64 are illustrated by:

Embodiment PA. A compound of Formula 1 in the Summary of the Disclosure wherein
R¹ is halogen, nitro or C₁-C₃ alkyl;
R² is cyano, nitro, halogen, C₁-C₄ alkyl or C₁-C₄ haloalkyl;
Q is C₁-C₆ alkyl, C₁-C₆ haloalkyl, C₁-C₆ alkylthio, C₁-C₆ haloalkylthio, C₁-C₆ alkylsulfinyl, C₁-C₆ alkylsulfonyl, C₁-C₆ alkoxy, C₁-C₆ haloalkoxy, C₂-C₅ alkylcarbonyl, C₂-C₅ alkoxycarbonyl, C₂-C₅ haloalkylcarbonyl, C₂-C₅ haloalkoxycarbonyl; or G;
G is phenyl substituted with 1 or 2 R³; or a 5- or 6-membered heterocyclic ring substituted with 1 or 2 R³; and
R³ is halogen, cyano, C₁-C₄ alkyl, C₁-C₄ haloalkyl, C₁-C₄ alkylsulfinyl, C₁-C₄ alkylsulfonyl, C₁-C₄ alkoxy or C₁-C₄ alkylthio.

Embodiment PB. A compound of Embodiment PA wherein
R¹ is F, Cl, Br, nitro or CH₃;
R² is C₁-C₄ alkyl or C₁-C₄ haloalkyl;
Q is C₁-C₆ alkyl, C₁-C₆ haloalkyl, C₁-C₆ haloalkylthio, C₁-C₆ haloalkoxy, C₂-C₅ alkylcarbonyl, C₂-C₅ alkoxycarbonyl or C₂-C₅ haloalkylcarbonyl; or G;
G is selected from

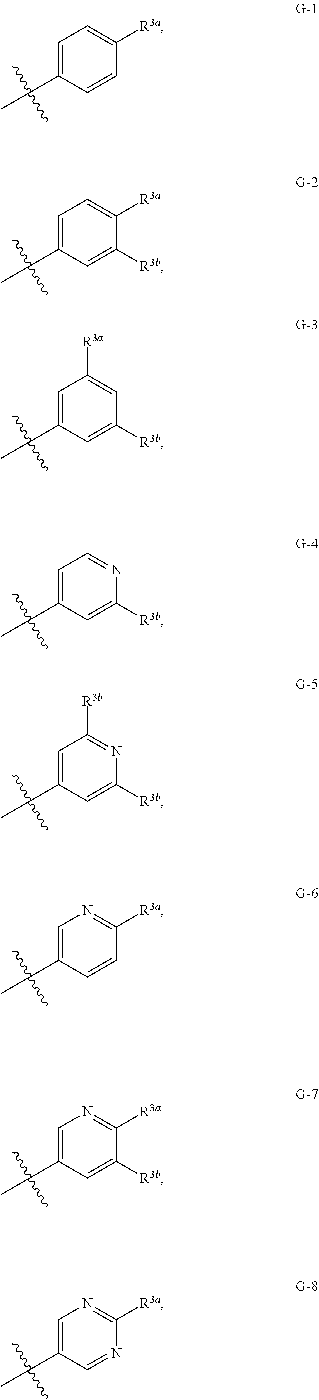

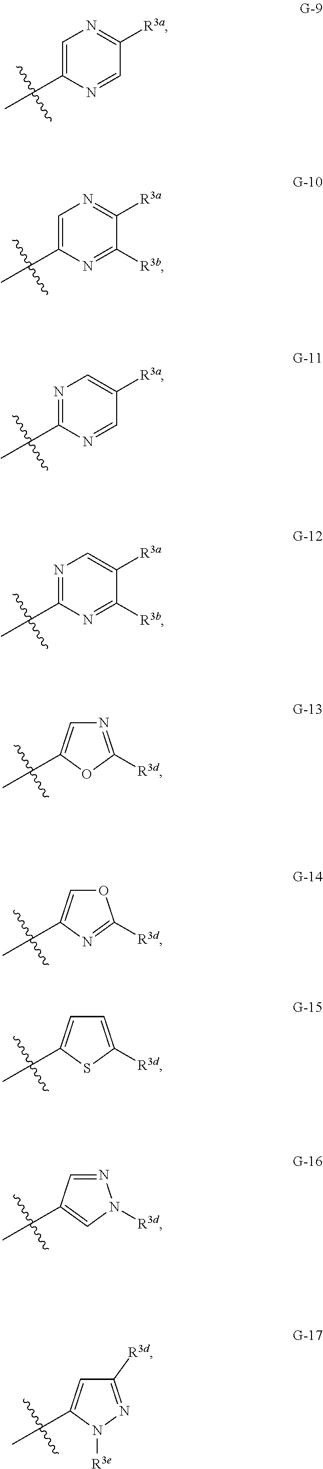

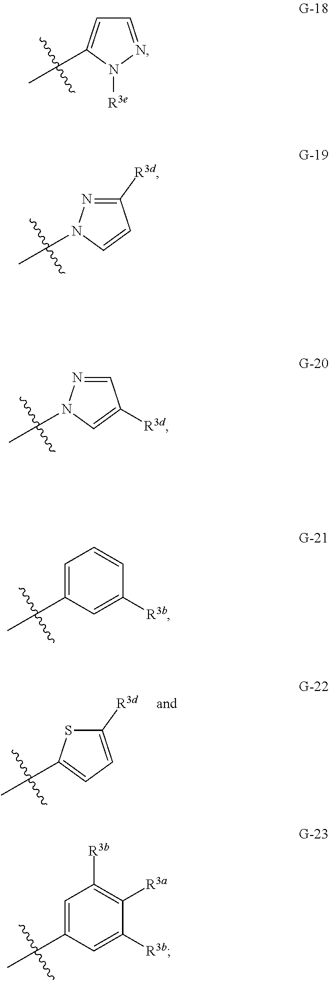

R³ is R^{3a}, R^{3b}, R^{3c}, R^{3d} or R^{3e}; and
R³ is halogen, C₁-C₃ alkyl or C₁-C₃ haloalkyl.

Embodiment PC. A compound of Embodiment PB wherein
R¹ is F, Cl, Br, nitro or CH₃;
R² is C₁-C₂ alkyl or C₁-C₂ haloalkyl;
Q is C₁-C₆ alkyl, C₃-C₆ haloalkyl, C₃-C₆ haloalkylthio, C₃-C₆ haloalkoxy, C₃-C₅ alkylcarbonyl, C₃-C₅ alkoxycarbonyl or C₃-C₅ haloalkylcarbonyl; or G;
G is selected from G-1, G-2, G-6, G-7, G-8 and G-15;
R^{3a} is Cl, F or CF₃;
R^{3b} is Cl or F; and
R^{3d} is Cl, CHF₂ or CF₃.

Embodiment PD. A compound of Embodiment PC wherein
R¹ is Cl, Br, nitro or CH₃;
R² is C₁-C₂ haloalkyl;
Q is C₁-C₆ alkyl or C₃-C₆ haloalkyl; or G;
G is G-1, G-2, G-6 and G-7; and
R^{3a} is Cl or CF₃.

Embodiment PE. A compound of Embodiment PD wherein
R¹ is Cl or Br;
R² is CHF₂, CF₃ or CF₂Cl;
Q is —CH₂CF₃, —CH₂CH₂CF₃—CH₂CH₂CH₂CF₃ or —OCH₂CH₂CH₂CF₃; or G;
G is G-1 and G-2.

Embodiment PF. A compound of Embodiment PD wherein
G is G-2;
R^{3a} is F; and
R^{3b} is Cl.

A specific Embodiment of a compound of Formula 1 is:

Embodiment PG. A compound of Formula 1 wherein
R¹ is Cl; R² is CF₃; Q is G; G is G-1; and R^{3a} is CF₃ (i.e. 5-chloro-2-[[3-(trifluoromethyl)-5-[4-(trifluoromethyl)phenyl]-1H-1,2,4-triazol-1-yl]methyl]pyrimidine (Compound 1).

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in crops such as wheat, barley, maize, soybean, sunflower, cotton, oilseed rape and rice, and specialty crops such as sugarcane, citrus, fruit and nut crops.

Also noteworthy as embodiments are herbicidal compositions of the present invention comprising the compounds of embodiments described above.

This invention also includes a herbicidal mixture comprising (a) a compound selected from Formula 1, N-oxides, and salts thereof, and (b) at least one additional active ingredient selected from photosystem II inhibitors (bi), AHAS inhibitors (b2), ACCase inhibitors (b3), auxin mimics (b4), EPSP synthase inhibitors (b5), photosystem I electron diverters (b6), PPO inhibitors (b7), GS inhibitors (b8), VLCFA elongase inhibitors (b9), auxin transport inhibitors (b10), PDS inhibitors (b11), HPPD inhibitors (b12), DXP synthase inhibitors (b13), HST inhibitors (b14), cellulose biosynthesis inhibitors (b15), DHODH inhibitors (b16), other herbicides including mitotic disruptors, organic arsenicals, asulam, bromobutide, cinflubrolin, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb (b17), and herbicide safeners (b18); and salts of compounds of (b1) through (b18).

“Photosystem II inhibitors” (b1) are chemical compounds that bind to the D-1 protein at the QB-binding niche and thus block electron transport from QA to QB in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The QB-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, amicarbazone, atrazine, bentazon, bromacil, bromofenoxim, bromoxynil, chlorbromuron, chloridazon, chlorotoluron, chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl, iodosulfuron-sodium, iofensulfuron, iofensulfuron-sodium, mesosulfuron-methyl, metazosulfuron (3-chloro-4-(5,6-dihydro-5-methyl-1,4,2-dioxazin-3-yl)-N-[[(4,6-dimethoxy-2-pyrimidinyl)amino]carbonyl]-1-methyl-1H-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo[1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, metproxybicyclone, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor and its methyl and ethyl esters and its sodium and potassium salts, aminopyralid, benazolin-ethyl, chloramben, clacyfos, clomeprop, clopyralid, dicamba, 2,4-D, 2,4-DB, dichlorprop, fluchloraminopyr, fluchloraminopyr-tefuryl, fluroxypyr, halauxifen, halauxifen-methyl, MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat, paraquat and 1-(2-carboxyethyl)-4-(2-pyrimidinyl)pyridazinium and salts and esters thereof. Of note is a photosystem I electron diverter selected from diquat and paraquat.

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, cinidon-ethyl, cyclopyranil, epyrifenacil, fluazolate, flufenoximacil, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione), tiafenacil and methyl 2-[2-[2-bromo-5-[3,6-dihydro-3-methyl-2,6-dioxo-4-(trifluoromethyl)-1(2H)-pyrimidinyl]-4-fluorophenoxy]phenoxy]-2-methoxyacetate, ethyl 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylate.

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl)butanoic acid) and bilanaphos.

“VLCFA elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)—N,N-diethyl-2-(1-naphthalenyloxy)propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl)phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid, diflufenican, fluridone, flurochloridone, flurtamone norflurzon and picolinafen.

“HPPD inhibitors” (b12) are chemical substances that inhibit the biosynthesis of synthesis of 4-hydroxyphenyl-pyruvate dioxygenase. Examples of HPPD inhibitors include benquinotrione, benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), bipyrazone, cyprafluone, fenpyrazone, fenquinotrione (2-[[8-chloro-3,4-dihydro-4-(4-methoxyphenyl)-3-oxo-2-quinoxalinyl]carbonyl]-1,3-cyclohexanedione), flusulfinam, iptriazopyrid, isoxachlortole, isoxaflutole, lancotrione, mesotrione, pyraquinate, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, tripyrasulfone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“Deoxy-D-Xylulose Phosphate Synthase (DXP synthase) inhibitors” (b13) isoxazolidinone class of herbicides is classified according to its inhibition of the deoxy-d-xylulose phosphate synthase (DXP synthase) which is a component of the carotenoid biosynthetic pathway. Examples of DXP synthase inhibitors include bixlozone, broclozone and clomazone. Of note is a DXP synthase inhibitor selected from bixlozone and clomazone.

“HST inhibitors” (b14) disrupt a plant's ability to convert homogentisate to 2-methyl-6-solanyl-1,4-benzoquinone, thereby disrupting carotenoid biosynthesis. Examples of HST inhibitors include haloxydine, pyriclor, 3-(2-chloro-3,6-difluorophenyl)-4-hydroxy-1-methyl-1,5-naphthyridin-2(1H1)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone. Another example of an HST inhibitor is 6-chloro-4-(2,7-dimethyl-1-naphthalenyl)-5-hydroxy-2-methyl-3(2H)-pyridazinone.

“Cellulose biosynthesis inhibitors” (b15) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-1H-inden-1-yl]-6-(1-fluoroethyl)-1,3,5-triazine-2,4-diamine), isoxaben and triaziflam.

“Dihydroorotate dehydrogenase (DHODH) inhibitors” (b16) inhibit dihydroorotate dehydrogenase of the de-novo pyrimidine biosynthetic pathway. The first two steps of de-novo pyrimidine biosynthesis occur in the chloroplast after which the product, N-carbamoyl aspartate, is shuttled to the cytoplasm where dihydroorotase resides, producing dihydroorotate, the DHODH substrate. Plant DHODH is a flavin-dependent enzyme residing on the outer surface of the inner mitochondrial membrane. Reducing equivalents pass from dihydroorotate via the tightly bound flavin cofactor to a ubiquinone acceptor molecule that subsequently exchanges with the ubiquinol pool of the membrane and ultimately links DHODH enzymatic activity with oxidative phosphorylation. The remaining steps of the pathway leading to pyrimidine nucleotides occur in the cytoplasm. DHODH inhibitors include a compound of Formula (b16A)

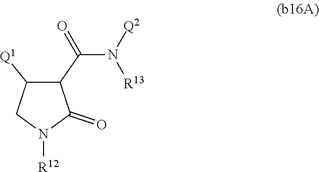

wherein
R¹² is H, C₁-C₆ alkyl, C₁-C₆ haloalkyl or C₄-C₈ cycloalkyl;
R¹³ is H, C₁-C₆ alkyl or C₁-C₆ alkoxy;
Q¹ is an optionally substituted ring system selected from the group consisting of phenyl, thienyl, pyridinyl, benzodioxolyl, naphthyl, naphthalenyl, benzofuranyl, furanyl, benzothiophenyl and pyrazolyl, wherein when substituted said ring system is substituted by 1 to 3 R¹⁴;
Q² is an optionally substituted ring system selected from the group consisting of phenyl, pyridinyl, benzodioxolyl, pyridinonyl, thiadiazolyl, thiazolyl, and oxazolyl, wherein when substituted said ring system is substituted by 1 to 3 R¹⁵;
each R¹⁴ is independently halogen, C₁-C₆ alkyl, C₁-C₆ haloalkyl, C₁-C₆ alkoxy, C₁-C₆ haloalkoxy, C₃-C₈ cyaloalkyl, cyano, C₁-C₆ alkylthio, C₁-C₆ alkylsulfinyl, C₁-C₆ alkylsulfonyl, SF₅, NHR¹⁷; or phenyl optionally substituted by 1 to 3 R¹⁶; or pyrazolyl optionally substituted by 1 to 3 R¹⁶;
each R¹⁵ is independently halogen, C₁-C₆ alkyl, C₁-C₆ haloalkyl, C₁-C₆ alkoxy, C₁-C₆ haloalkoxy, cyano, nitro, C₁-C₆ alkylthio, C₁-C₆ alkylsulfinyl, C₁-C₆ alkylsulfonyl;
each R¹⁶ is independently halogen, C₁-C₆ alkyl or C₁-C₆ haloalkyl;
R¹⁷ is C₁-C₄ alkoxycarbonyl.

In one Embodiment wherein “DHODH inhibitors” (b16) also include a compound of Formula (b16A), it is preferred that R¹² is H or C₁-C₆ alkyl; more preferably R¹² is H or methyl. Preferrably R¹³ is H. Preferably Q¹ is either a phenyl ring or a pyridinyl ring, each ring substituted by 1 to 3 R¹⁴; more preferably Q¹ is a phenyl ring substituted by 1 to 2 R¹⁴. Preferably Q² is a phenyl ring substituted by 1 to 3 R¹⁵; more preferably Q² is a phenyl ring substituted by 1 to 2 R¹⁵. Preferably each R¹⁴ is independently halogen, C₁-C₄ alkyl, C₁-C₃ haloalkyl, C₁-C₃ alkoxy or C₁-C₃ haloalkoxy; more preferably each R¹⁴ is independently chloro, fluoro, bromo, C₁-C₂ haloalkyl, C₁-C₂ haloalkoxy or C₁-C₂ alkoxy. Preferrably each R¹⁵ is independently halogen, C₁-C₄ alkyl, C₁-C₃ haloalkoxy; more preferably each R¹⁵ is independently chloro, fluoro, bromo, C₁-C₂ haloalkyl, C₁-C₂ haloalkoxy or C₁-C₂ alkoxy. Specifically preferred as “DHODH inhibitors” (b16) include any one of the following (b16A-1) through (b16A-17):
N-(2-fluorophenyl)-2-oxo-4-[3-(trifluoromethyl)phenyl]-3-pyrrolidinecarboxamide (b-16A-1);
N-(2,3-difluorophenyl)-2-oxo-4-[3-(trifluoromethyl)phenyl]-3-pyrrolidinecarboxamide (b16A-2);
2-oxo-4-[3-(trifluoromethyl)phenyl]-N-(2,3,4-trifluorophenyl)-3-pyrrolidinecarboxamide (b16A-3);
N-(2-fluorophenyl)-1-methyl-2-oxo-4-[3-(trifluoromethyl)phenyl]-3-pyrrolidinecarboxamide (b16A-4);
N-(2-fluorophenyl)-2-oxo-4-[4-(trifluoromethyl)phenyl]-3-pyrrolidinecarboxamide (b16A-5);
N-(2-fluorophenyl)-1-methyl-2-oxo-4-[4-(trifluoromethyl)phenyl]-3-pyrrolidinecarboxamide (b16A-6);
N-(2,3-difluorophenyl)-2-oxo-4-[4-(trifluoromethyl)phenyl]-3-pyrrolidinecarboxamide (b16A-7);
N-(2,3-difluorophenyl)-1-methyl-2-oxo-4-[4-(trifluoromethyl)phenyl]-3-pyrrolidinecarboxamide (b16A-8);
2-oxo-4-[4-(trifluoromethyl)phenyl]-N-(2,3,4-trifluorophenyl)-3-pyrrolidinecarboxamide (b16A-9);
N-(2-fluorophenyl)-4-(4-fluorophenyl)-1-methyl-2-oxo-3-pyrrolidinecarboxamide (b16A-10);
N-(2,3-difluorophenyl)-4-(3,4-difluorophenyl)-2-oxo-3-pyrrolidinecarboxamide (b16A-11);
4-(3,4-difluorophenyl)-N-(2-fluorophenyl)-2-oxo-3-pyrrolidinecarboxamide (b16A-12);
N-(2,4-difluorophenyl)-4-(3,5-difluorophenyl)-2-oxo-3-pyrrolidinecarboxamide (b16A-13);
N-(2,3-difluorophenyl)-4-[3-(1-methylethyl)phenyl]-2-oxo-3-pyrrolidinecarboxamide (b16A-14);
N-(2,3-difluorophenyl)-2-oxo-4-[6-(trifluoromethyl)-3-pyridinyl]-3-pyrrolidinecarboxamide (b16A-15);
(3S,4S)—N-(2-fluorophenyl)-1-methyl-2-oxo-4-[3-(trifluoromethyl)phenyl]-3-pyrrolidinecarboxamide (b16A-16) and
(3S,4R)—N-(2,3-difluorophenyl)-1-methyl-4-[1-methyl-5-(trifluoromethyl)-1H-pyrazol-3-yl]-2-oxo-3-pyrrolidinecaboxamide (b16A-17).

An example of a DHODH inhibitor is tetflupyrolimet.

“Other herbicides” (b17) include herbicides that act through a variety of different modes of action such as mitotic disruptors (e.g., flamprop-M-methyl, flamprop-M-isopropyl, icafolin and icafolin-methyl), organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b16) or act through a combination of modes of action listed above. Examples of other herbicides include aclonifen, asulam, amitrole, bromobutide, cinmethylin, cumyluron, cyclopyrimorate (6-chloro-3-(2-cyclopropyl-6-methylphenoxy)-4-pyridazinyl 4-morpholinecarboxylate), daimuron, difenzoquat, etobenzanid, fluometuron, flurenol, fosamine, fosamine-ammonium, dazomet, dymron, ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb and 5-[[(2,6-difluorophenyl)methoxy]methyl]-4,5-dihydro-5-methyl-3-(3-methyl-2-thienyl)isoxazole. Other herbicides also include a compound of Formula (b18A),

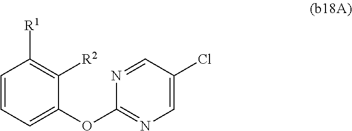

wherein R¹ is Cl, Br or CN; and R² is C(═O)CH₂CH₂CF₃, CH₂CH₂CH₂CH₂CF₃ or 3-CHF₂-isoxazol-5-yl. An example of an other herbicide is rimisoxafen.

“Herbicide safeners” (b18) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

The compounds of Formula 1 can be prepared by general methods known in the art of synthetic organic chemistry. One or more of the following methods and variations as described in Schemes 1-14 can be used to prepare the compounds of Formula 1. The definitions of R¹, Q and G in the compounds of Formulae 1-20 below are as defined above in the Summary of the Invention unless otherwise noted. Compounds of Formulae 1a-1i are various subsets of the compounds of Formula 1, and all substituents for Formulae 1a-1i are as defined above for Formula 1 unless otherwise noted.

As shown in Scheme 1, a compound of Formula 1 can be prepared by nucleophilic substitution by heating a compound of Formula 2, (wherein Q is defined in the Summary of the Invention) in a suitable solvent, such as acetonitrile, tetrahydrofuran or N,N-dimethylformamide in the presence of a base (e.g., potassium or cesium carbonate), with a compound of Formula 3, (where R¹ is defined in the Summary of the Invention and LG is a leaving group, such as halogen). The reaction is typically conducted at temperatures ranging from ambient to 90° C.

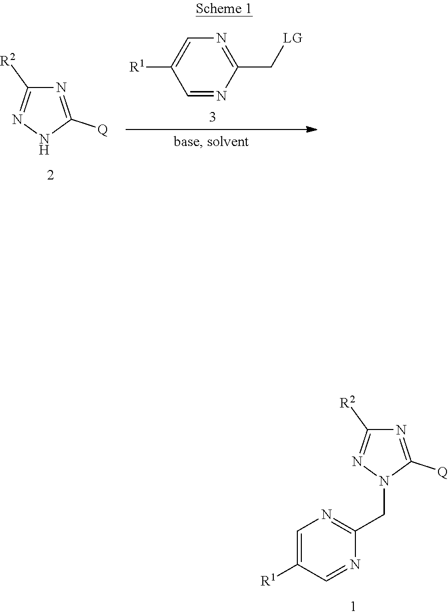

As shown in Scheme 2, a compound of Formula 1a, (a compound of Formula 1 wherein Q is defined as G in the Summary of the Invention) can be prepared by using “C—H arylation” chemistry. A compound of Formula 4 is reacted with a compound of Formula 3 with conditions analogous to the method described in Scheme 1 to produce a compound of Formula 5. The alkylated compound of Formula 5 is then arylated using C—H activation methods found in Synlett, 2020, 31, 1015 as illustrated in Example 1, Step B and Example 3, Step A.

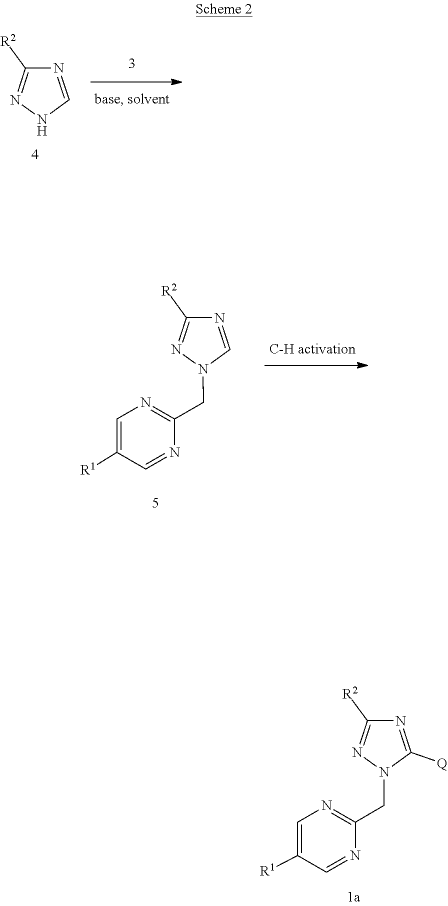

Scheme 3 illustrates how a compound of Formula 1 can be prepared by reaction of a compound of Formula 6 (where R¹ and R² are defined in the Summary of the Invention and X is halogen) with an organometallic compound of Formula 7a (i.e. Q-M¹, where Q is defined in the Summary of the Invention) under transition metal catalyzed cross-coupling reaction conditions with palladium or nickel catalysts. In this method a compound of Formula 7a, M¹ can be organoboronic acids or esters (M¹ is B(OH)₂ or B(Pin), organotin reagents (M¹ is Sn(n-Bu)₃, Sn (Me)₃), Grignard reagents (M¹ is MgBr or MgCl) or organozinc reagents (M¹ is ZnBr or ZnCl). Suitable metal catalysts include, but are not limited to, palladium(II) acetate, palladium(II) chloride, tetrakis (triphenylphosphine) palladium, bis(triphenylphosphine)palladium(II) dichloride, dichloro[1,1-bis(diphenylphosphino)ferrocene]palladium(II) dichloromethane complex, (2-dicyclohexylphosphino-2′,4′,6′-triisopropyl-1,1′-biphenyl)[2-(2′amino-1,1,′-biphenyl]palladium(II)chloride (X-Phos palladacycle), bis(triphenylphosphine)dichloronickel(II). It is understood by one skilled in the art that depending on M¹, addition of a ligand (substituted phosphine or bisphosphinoalkane), or the presence of bases such as alkali carbonate, potassium phosphate, tertiary amine or alkali fluoride in solvents such as dioxane, 2-methyl tetrahydrofuan, 1,2-dimethoxyethane, toluene, N,N-dimethylformamide and water preferably under a nitrogen atmosphere may be required for optimal reaction conditions. The reaction is typically carried out at temperatures ranging from ambient to the boiling point of the solvent. The reaction can also be carried out at temperatures above the solvent boiling point by using a pressurized vessel, such as a microwave reactor or Fisher-Porter tube. For reviews of these various types of reactions see: E. Negishi, Handbook of Organopalladium Chemistry for Organic Synthesis, John Wiley and Sons, Inc., New York, 2002; N. Miyaura, Cross-Coupling Reactions: A Practical Guide, Springer, New York, 2002; H. C. Brown et al, Organic Synthesis via Boranes, Vol. 3, Aldrich Chemical Co., Milwaukee, WI, 2002; Suzuki et al, Chemical Review 1995, 95, 2457-2483. Alternatively, a compound of Formula 7a is commercially available or can be prepared by methods known in the literature. A compound of Formula 4b can be reacted with a compound of Formula 3 with conditions analogous to the method described in Scheme 1 to produce a compound of Formula 6 (where X is a leaving group). See Synthesis Example 7 for examples of these methods.

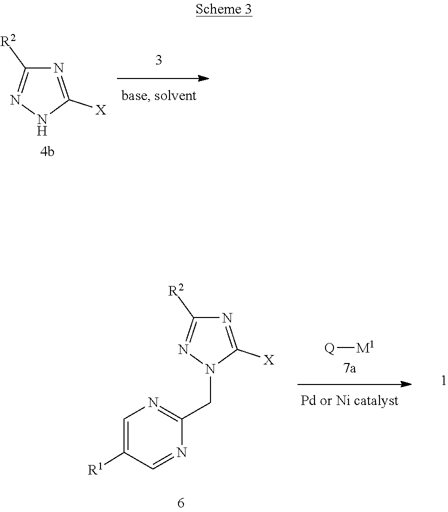

Alternatively, as shown in Scheme 4, a compound of Formula 6 (a compound of Formula 6 where X is halogen) can also be prepared by treating compounds of Formula 5 with sterically hindered 2,2,6,6-tetramethyl piperidyl bases (TMP-ZnCl LiCl or TMP-MgCl LiCl) at temperatures ranging from −70° C. to ambient temperature and reacting with an electrophile such as iodine or bromine in a solvent such as tetrahydrofuran.

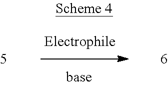

As shown in Scheme 5, a compound of Formula 5 can be directly zincated with zinc bases (eg., TMP-ZnCl LiCl or 2,2,6,6-tetramethylpiperidinylzinc chloride lithium chloride complex) to prepare a compound of Formula 5a (in situ) and then subsequently reacted with a compound of Formula 7b (i.e. Q¹-X where X is Br or I) to provide a compound of Formula 1b (wherein Q is a carbon-linked Q). Examples of Negishi type coupling conditions using Pd(OAc)₂ and SPhos as a preferred catalyst/ligand system with a compound of Formula 7b, Q¹-X, are found in Org. Lett. 2020, 22, 1899-1902. Another particularly useful catalyst/ligand combination for Negishi coupling reactions is tris(dibenzylideneacetone)dipalladium(0) and tri(2-furyl) phosphine. The compound of Formula 5a can be used in copper catalyzed cross-couplings with the addition of CuCN—LiCl and a compound of Formula 7b to prepare a compound of Formula 1, (wherein Q is a C-linked aromatic ring or carbonyl-containing aliphatic group).

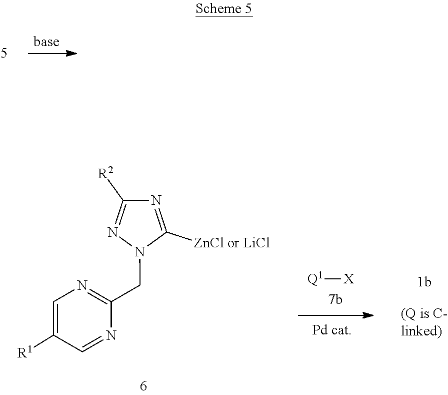

As shown in Scheme 6, a compound of Formula 8 can be alkylated with a compound of Formula 9, (where LG is a leaving group, such as halogen) in the presence of a compound of Formula 3 and a base (eg., potassium or cesium carbonate) in a suitable solvent, such as acetonitrile, tetrahydrofuran or N,N-dimethylformamide to prepare a compound of Formula 10 (where R⁴ is C₁-C₆ alkyl). The reaction is typically conducted at temperatures from ambient temperature to 60° C. A compound of Formula 1c (i.e. where Q is C₁-C₆ alkylthio) can be prepared from a compound of Formula 10 using methods analogous to the method described in Scheme 1. See Synthesis Example 5 for a method to prepare a compound of Formulae 10 and 1c.

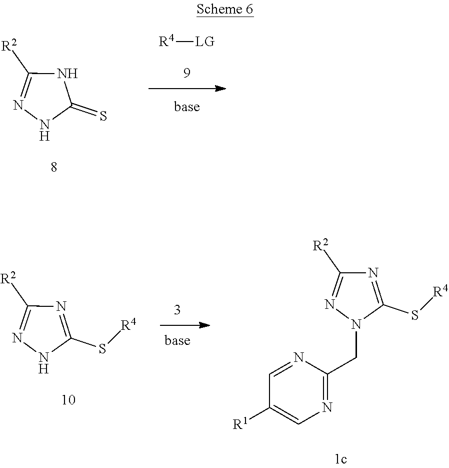

As shown in Scheme 7 the compounds of Formulae 1d (where m is 1) or 1e (where m is 2) are prepared from the oxidation of a compound of Formula 1c with an oxidizing reagent (e.g., MCPBA or Oxone®) in solvents like dichloromethane or acetone at temperatures that range from zero to ambient to give the sulfone (m is 2, where Q is C₁-C₆ alkylsulfonyl) as illustrated in Example 5, Step C or sulfoxide (m is 1, where Q is C₁-C₆ alkylsulfinyl). A compound of Formula if (where Q is N-linked, e.g., where Q is G, and G is G-19 or G-20) is then prepared by nucleophilic substitution with heating of a compound of Formula 1d with a compound of Formula 13 (Q¹-NH containing) in the presence of a base (selected from sodium hydride, potassium tert-butoxide, cesium carbonate and potassium carbonate) in a solvent such tetrahydrofuran, N,N-dimethylformamide or dimethylsulfoxide with stirring in a range from ambient to heating to 180° C., including in under microwave conditions by methods reported in the literature (Org. Lett. 2021, 23(15), 5761-5765 and J. Org. Chem. 2008, 73(17), 6816-6823). See Example 5, Step D for an example of these nucleophilic substitution reactions.

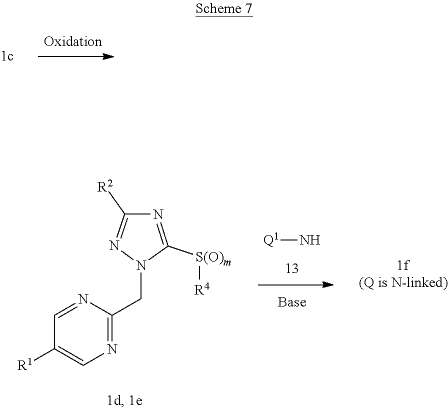

As shown in Scheme 8, a compound of Formula if (where Q is N-linked, e.g., when Q is G, and G is G-19 or G-20) can also be prepared by nucleophilic displacement of compounds of Formula 6a (where X is Br, Cl or I; preferably Br or I; most preferrably Br; or most preferably I) by treating compounds of Formula 13 (wherein Q¹-NH containing) in the presence of bases (eg., sodium hydride, potassium tert-butoxide, cesium or potassium carbonate) in solvents like tetrahydrofuran, N,N-dimethyl formamide, dimethylsulfoxide at a temperature range of ambient to 180° C., including in the microwave (See WO2012064559, Adv. Synth. & Catal. 2023, 365(12), 2013-2017, ACS Med. Chem. Lett. 2010, 1(9), 526-529 and WO2009134750 for examples of the nucleophilic substitution reactions).

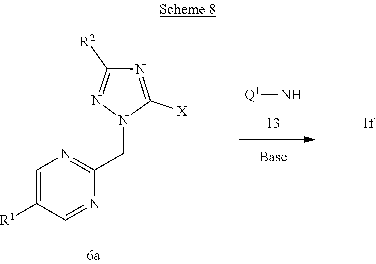

As shown in Scheme 9, a compound of Formula 1g (where Q is other than G) can be prepared by first N-protecting a compound of Formula 4 with a compound of Formula 11, (PG-LG, e.g., N,N-dimethylsulfamoyl chloride, trimethylsilylethoxymethyl chloride, triisopropylsily chloride, p-methoxybenzyl) in the presence of a base (eg., potassium or sodium carbonate, triethylamine) in solvents such as N,N-dimethylformamide, acetonitrile, dichloromethane typically at temperatures ranging from ambient to 100° C. to give a compound of Formula 12. A compound of Formula 12 can be metalated with a base (eg., n-butyllithium, lithium diisopropylamide, lithium tetramethylpiperidide) in solvents tetrahydrofuran or diethyl ether at temperatures typically below −70° C. and quenched with either N,N-dimethylformamide to give the aldehyde of Formula 14 or compounds of Formula 13 (R⁴—CHO; R⁴ is C₁-C₆ alkyl) to yield the alcohol, a compound of Formula 16. Aldehydes of Formula 15 can be reacted with the Grignard or lithium reagents of Formula 14 (R⁴—MgX or R⁴—Li) to prepare a compound of Formula 20. A compound of Formula 16 can be oxidized with an oxidation reagent of Formula 17 (eg., Dess-Martin Periodane or Jones reagent) in solvents such as dichloromethane or acetone to prepare a compound of Formula 18. A compound of Formula 18 can then be deprotected under acidic conditions (eg., trifluoroacetic acid or HCl in dioxanes or water) at temperatures ranging from ambient to 100° C. Once deprotected a compound of Formula 19 can be reacted in a similar method to that described in Scheme 1 to prepare a compound of Formula 1g (wherein Q is C₂-C₅ alkylcarbonyl, C₂-C₅ alkoxycarbonyl, C₂-C₅ haloalkylcarbonyl or C₂-C₅ haloalkoxycarbonyl).

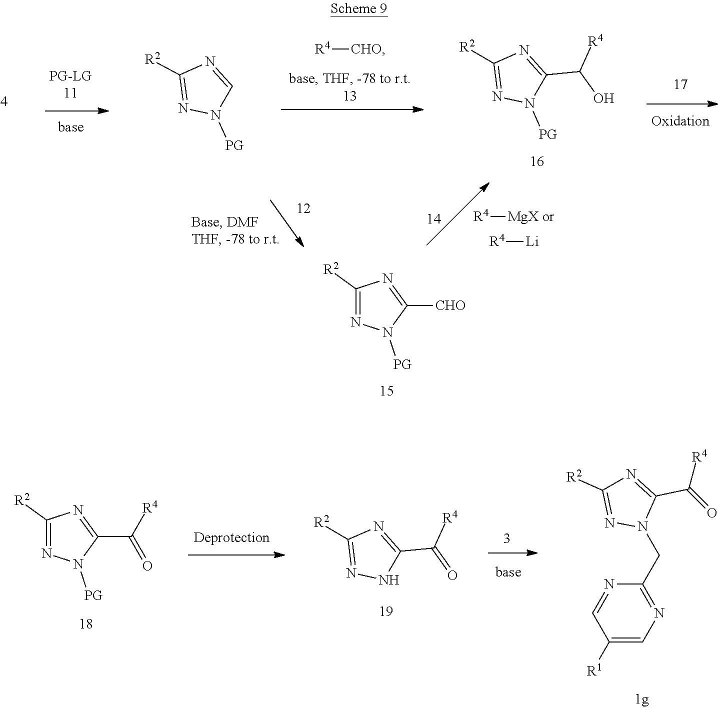

Compounds of Formula 1h (where R²═CHF₂) in Scheme 10 can be prepared from a compound of Formula 6d, (where R⁵ is a lower alkyl or H; and X is a leaving group). A compound of Formula 6d is reduced with reducing reagents (eg., sodium borohydride in ethanol at 0° C. to ambient temperature; diisobutyl aluminum hydride or lithium aluminum hydride in tetrahydrofuran at −70° C. to ambient temperature) to prepare the alcohol of Formula 6e. A compound of Formula 6e is then oxidized with oxidizing reagents (eg., manganese(IV) oxide or pyridinium chlorochromate, pyridinium dichromate) at ambient to reflux temperatures to the aldehyde of Formula 6f. Difluoro methylation of compounds of Formula 6f can be prepared with fluorinating reagents (eg., DAST®, diethylaminosulfur trifluoride or Deoxyfluor®, bis(2-methoxyethyl) aminosulfur trifluoride) in dichloromethane at zero degrees to give a compound of Formula 6g. Compounds of Formula 1h, (wherein R² is CHF₂) are prepared from a compound of Formula 6g in a similar manner to the methods describe in Schemes 3 and 8.

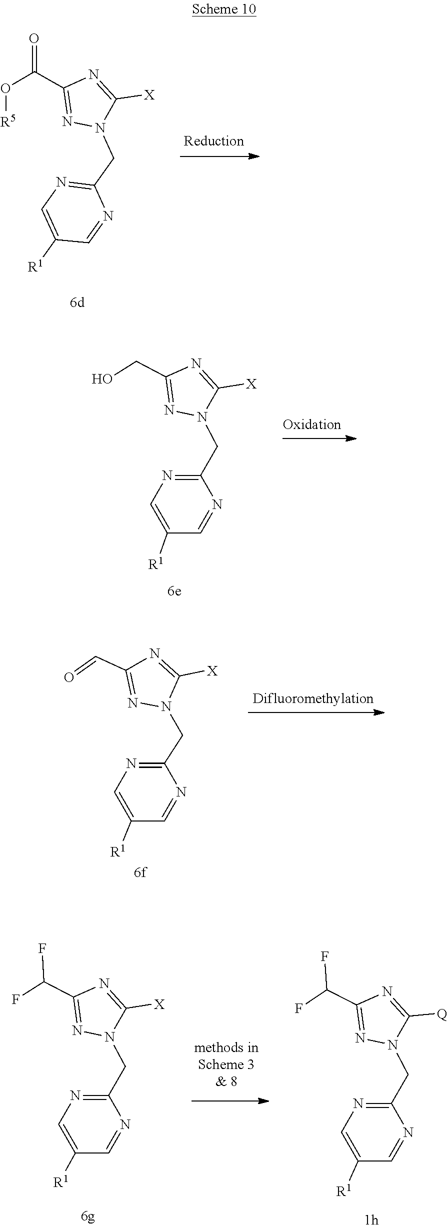

Compounds of Formula 1h (where R²═CHF₂) can also be prepared from intermediates of Formula 1i, where R⁵ is lower alkyl or H, and Q is defined in the Summary of the Invention in a similar manner to the methods in Scheme 10.

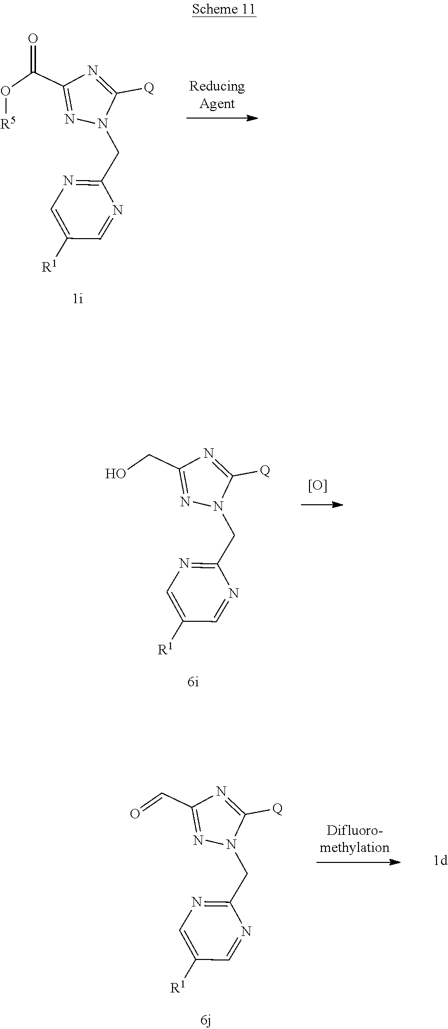

The compounds of Formulae 2 and 4 as shown below in Scheme 12 are commercially available or can be prepared from methods described in Science of Synthesis, vol: 13, pages: 603-639, 2004. Compounds of Formula 2, (particularly where Q is alkyl) can be prepared by Minisci reactions of a compound of Formula 4 with Q-CO₂H (where Q is alkyl or haloalkyl) using the methods in Med. Chem. Comm. 2011, 2, 1135-1161.

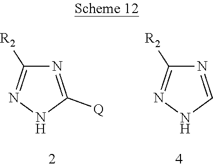

A compound of Formula 3a (i.e. where both X's are halogen) can be prepared by the method shown in Scheme 13 via radical bromination of 5-halo-2-methylpyrimidine (20) with radical initiators such as benzoyl peroxide or AIBN with a halogenating reagent like N-halosuccimimides (NXS where X is Cl, Br or I) in solvents such as carbon tetrachloride or chloroform with heating at a temperature of from 70 to 100° C.

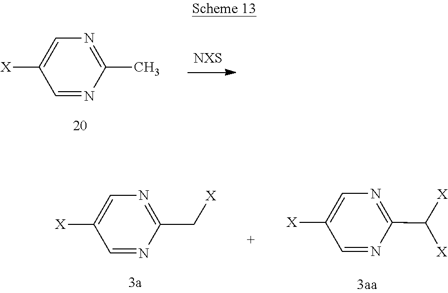

Alternatively, a compound of Formula 3a can be prepared by treatment of a 2-cyano-5-halopyrimidine compound of Formula 21 with hydrogen chloride in a lower alkyl alcohol (methyl or ethyl, R⁴—OH) to form the ester product, 22 as shown in Scheme 14. Reduction with and suitable reducing agent (i.e. similar to those listed for Scheme 10: 6a to 24) gives the alcohol 3b. 3b can be halogenated using reagents such as thionyl chloride or phosphorous tribromide in chlorinated solvents (chloroform or dichloromethane) at temperatures from zero to ambient temperature to heating to 60° C. to obtain the 5-halo-2-(halomethyl) pyrimidine 3a (where X is halogen, preferrably Cl or Br). For examples of these types of reaction conditions see WO 2013/142269.

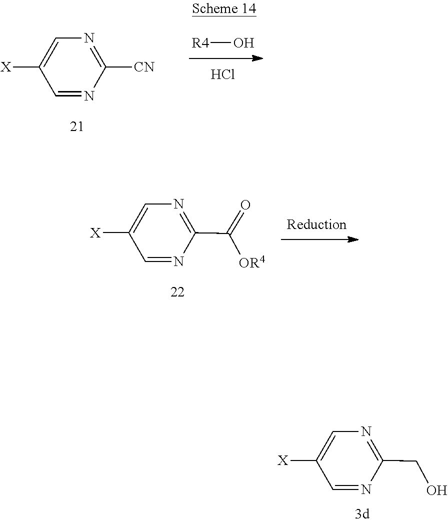

Scheme 15 outlines another method the method as described in Dyes and Pigments 2018, 159, 619-636 to cyclize the pyrimidine ring to produce intermediate 35, 5-chloro-2-(methoxymethyl)pyrimidine. Compound 35 is converted to the alcohol 3b using boron tribromide in dichloromethane at 0° C. Compound 3b is converted to 3a (where X is chloro or bromo) using similar conditions as those in Scheme 13 above. Finally, stirring a compound of 3a where X is Cl in hydrogen chloride in an organic solvent such as ethyl acetate obtains 3a′ as the hydrogen chloride salt.

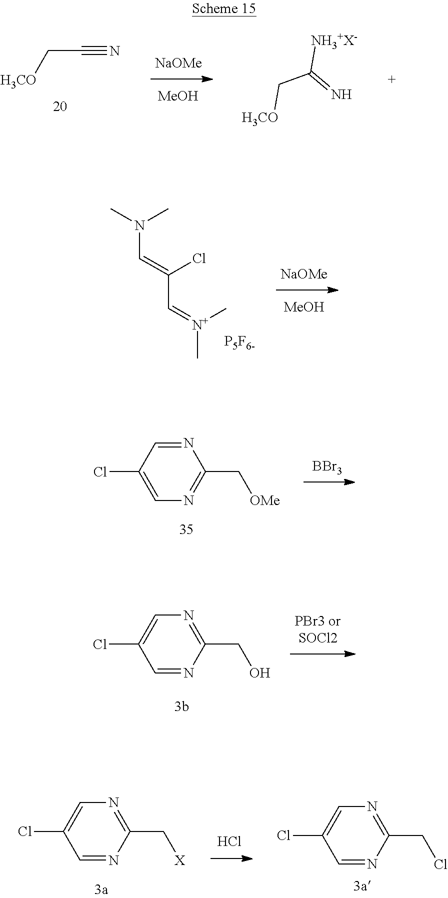

Scheme 16 outlines an alternative synthesis sequence that can be used to prepare a compound of Formula 1.

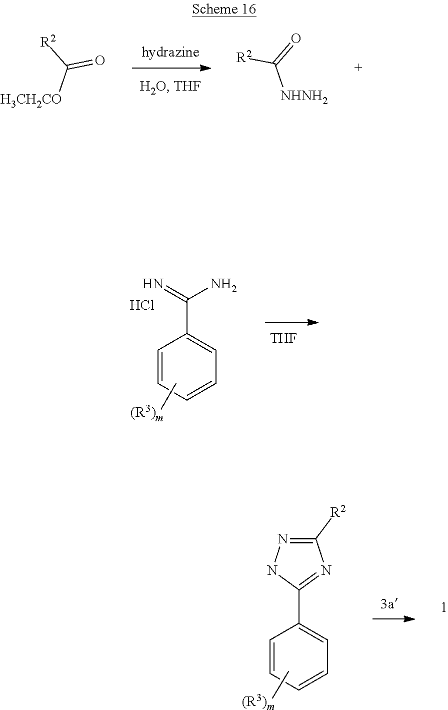

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above Schemes in an order other than that implied by the particular order presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane at 500 MHz in CDCl₃ unless otherwise indicated. ¹⁹F NMR spectra are reported in ppm downfield from CFCl₃ at 471 MHz in CDCl₃ unless otherwise indicated; “s” means singlet, “d” means doublet, “t” means triplet, “m” means multiplet, “dd” means doublet of doublets, “br s” means broad singlet, “br d” means broad doublet, and “bp” means broad peak. Mass spectra (MS) are reported as the molecular weight of the highest isotopic abundance parent ion (M+1) formed by addition of H+ (molecular weight of 1) to the molecule, or (M−1) formed by the loss of H+ (molecular weight of 1) from the molecule, observed by using liquid chromatography coupled to a mass spectrometer (LCMS) using either atmospheric pressure chemical ionization (AP+) where “amu” stands for unified atomic mass units. MPLC means medium pressure liquid chromatography.

### Synthesis Example 1

### Preparation of 5-chloro-2-[[3-(trifluoromethyl)-5-[4-(trifluoromethyl)phenyl]-1H-1,2,4-triazol-1-yl]methyl]pyrimidine (Compound 1)

### Step A: Preparation of 5-chloro-2-[[3-(trifluoromethyl)-1H-1,2,4-triazol-1-yl]methyl]pyrimidine

3-(Trifluoromethyl)-1H-1,2,4-triazole (200 mg, 1.46 mmol) was dissolved in acetonitrile (4.5 mL) under a nitrogen atmosphere. To this mixture was added potassium carbonate (0.605 g, 4.38 mmol) then 5-chloro-2-(chloromethyl)-pyrimidine hydrochloride (1:1) (0.349 g, 1.75 mmol) followed by de-ionized water (0.04 mL). The reaction mixture was stirred at ambient temperature overnight. An aliquat was removed from the reaction mixture for mass spectral analysis, which revealed the presence of the desired product material (ES⁺=264, 1×Cl, ES⁻=262). The reaction mixture was diluted with water and extracted with ethyl acetate (3×). The combined organic layers were washed with water, brine, dried over MgSO₄ then filtered. The filtrate was concentrated to provide 0.4 g oil which was purified using a 12 g (silica gel) MPLC column using 0 to 55% ethyl acetate in hexanes to provide the title compound as clear oil (0.35 g).

¹H NMR δ ppm 5.65 (s, 2H), 8.39 (s, 1H), 8.69 (s, 2H).

¹⁹F NMR δ ppm −65.19 (s, 1×CF₃).

### Step B: Preparation of 5-chloro-2-[[3-(trifluoromethyl)-5-[4-(trifluoromethyl)phenyl]-1H-1,2,4-triazol-1-yl]methyl]pyrimidine

5-Chloro-2-[[3-(trifluoromethyl)-1H-1,2,4-triazol-1-yl]methyl]pyrimidine (i.e. the product of Example 1, Step A) (100 mg, 0.379 mmol) was combined with 4-iodobenzotrifluoride (0.13 g, 0.48 mmol) in p-dioxane (2.75 mL) in a microwave vial. To this mixture was added palladium(II) acetate (6 mg), triphenyl phosphine (11 mg), copper(I) iodide (0.15 g) and 1,8-diazabicyclo[5.4.0]undec-7-ene (0.12 g, 0.79 mmol). The vial was capped, and the mixture was stirred for 3 min, then placed in the microwave. The mixture was heated in the microwave at 140° C. for 2 h. An aliquat was removed from the reaction mixture for mass spectral analysis, which revealed the presence of the desired product material (dual ES⁺=408 & ES⁻=406). The reaction mass was diluted with water and extracted with ethyl acetate (3×). The combined organic layers were washed with brine, dried over MgSO₄, filtered, and concentrated to 0.15 g of an oil which was purified using a (silica gel) 12 g MPLC column using a 0 to 55% ethyl acetate/hexanes gradient to isolate a solid (0.1 g) which was suspended in hexanes and filtered provide the title compound as a solid (39 mg). Mass Spec. (dual) ES⁺=408 and ES⁻=406 (both 1×Cl pattern).

¹H NMR δ ppm 5.68 (s, 2H), 7.77 (d, 2H), 7.95 (br d, 2H), 8.70 (s, 2H).

¹⁹F NMR δ ppm −65.30 (s, 1×CF₃), −63.09 (s, 1×CF₃).

### Synthesis Example 2

### Preparation of 5-chloro-2-[[5-(4-chlorophenyl)-3-(trifluoromethyl)-1H-1,2,4-triazol-1-yl]methyl]pyrimidine (Compound 2)

### Step A: Preparation of 5-chloro-2-[[5-(4-chlorophenyl)-3-(trifluoromethyl)-1H-1,2,4-triazol-1-yl]methyl]pyrimidine (Compound 2)

5-Chloro-2-[[3-(trifluoromethyl)-1H-1,2,4-triazol-1-yl]methyl]pyrimidine (100 mg, 0.379 mmol) and 1-chloro-4-iodobenzene were combined in p-dioxane (2.75 mL) in a microwave vial. Palladium(II) acetate (6 mg) was added, followed by triphenylphosphine (11 mg), copper(I) iodide (0.15 g) 1,8-diazabicyclo[5.4.0]undec-7-ene (0.12 g, 0.79 mmol) then the vial was capped and stirred for 5 min at ambient temperature. The vial and contents were placed in the microwave and heated to 140° C. for 2 h. The reaction was diluted with water then extracted with ethyl acetate (3×). The combined organic layers were washed with brine, dried over MgSO₄, filtered and concentrated to provide a solid (0.17 g). This solid was purified through a (silica gel) 12 g MPLC column using a 0 to 20% ethyl acetate in hexanes gradient to provide the title compound as a solid (39 mg). Mass Spec (dual ES⁺=374 and ES⁻=372 (Cl₂ pattern).

¹H NMR δ ppm 5.66 (s, 2H), 7.44-7.55 (m, 2H), 7.66-7.86 (m, 2H), 8.59-8.79 (m, 2H).

¹⁹F NMR δ ppm −65.32 (s, 1×CF₃).

### Synthesis Example 3

### Preparation of 5-chloro-2-[[3-(trifluoromethyl)-5-[6-(trifluoromethyl)-3-pyridinyl]-1H-1,2,4-triazol-1-yl]methyl]pyrimidine (Compound 4)

### Step A: Preparation of 5-chloro-2-[[3-(trifluoromethyl)-5-[6-(trifluoromethyl)-3-pyridinyl]-1H-1,2,4-triazol-1-yl]methyl]pyrimidine

5-Chloro-2-[[3-(trifluoromethyl)-1H-1,2,4-triazol-1-yl]methyl]pyrimidine (i.e. from Example 1, Step A) (100 mg, 0.379 mmol) was combined with 5-iodo-2-(trifluoromethyl)pyridine (0.13 g, 0.48 mmol) in p-dioxane (2.75 mL) in a microwave vial. To this mixture was added palladium(II) acetate (6 mg), triphenylphosphine (11 mg), copper(I) iodide (0.15 g) and 1,8-diazabicyclo[5.4.0]undec-7-ene (0.12 g, 0.79 mmol), then capped. The mixture was stirred for 3 min, then placed in the microwave and heated at 140° C. for 2 h. An aliquat was removed from the reaction mixture for mass spectral analysis, which revealed the presence of the desired product material (dual ES⁺=409 & ES⁻=407). The reaction mixture was diluted with water, then extracted with ethyl acetate (3×). The combined organic layers were washed with brine, dried over MgSO₄, filtered then concentrated to provide 0.13 g of an oil. The oil was purified by silica gel chromatography with a 12 g MPLC column using a 0 to 55% ethyl acetate in hexanes gradient to obtain the title compound as a solid (80 mg). Mass Spec (dual) ES⁺=409 & ES⁻=407 (both 1×Cl pattern).

¹H NMR δ ppm 5.70 (s, 2H), 7.86 (dd, 1H), 8.42 (dd, 1H), 8.71 (s, 2H), 9.20 (d, 1H).

¹⁹F NMR δ ppm −68.23 (s, 1×CF₃), −65.33 (s, 1×CF₃).

### Synthesis Example 4

### Preparation of 5-chloro-2-[[3-methyl-5-[4-(trifluoromethyl)phenyl]-1H-1,2,4-triazol-1-yl]methyl]pyrimidine (Compound 3)

### Step A: Preparation of 5-chloro-2-[(3-methyl-1H-1,2,4-triazol-1-yl)methyl]pyrimidine

Dissolved 3-methyl-1H-1,2,4-triazole (200 mg, 2.41 mmol) in acetonitrile (7 mL, 0.340 M) under a nitrogen atmosphere. Added potassium carbonate (0.998 g, 7.22 mmol) then 5-chloro-2-(chloromethyl)-pyrimidine hydrochloride (1:1) (0.576 g, 2.89 mmol). The reaction mixture was heated to 80° C. for 16 h, then diluted with water and extracted with ethyl acetate (3×). The combined organic layers were washed with water, brine, dried over MgSO₄, filtered, and concentrated to provide and oil (0.59 g). The oil was purified using a (silica gel) 12 g MPLC column with a gradient of 0 to 55% ethyl acetate in hexanes to obtain a clear oil (0.59 g) which crystallized. A white solid was filtered from hexanes to obtain the title compound as a white solid (0.31 g). Mass Spec (Dual) ES⁺=210 (1×Cl) ES⁻=208.

¹H NMR δ ppm 2.40 (s, 3H), 2.51 (s, 3H), 5.50 (s, 2H), 5.51 (s, 3H), 7.83 (s, 1H), 8.15 (s, 1H), 8.67 (d, 3H)—a mixture of the title compound and starting material.

### Step B: Preparation of 5-chloro-2-[[3-methyl-5-[4-(trifluoromethyl)phenyl]-1H-1,2,4-triazol-1-yl]methyl]pyrimidine

Dissolved 5-chloro-2-[(3-methyl-1H-1,2,4-triazol-1-yl)methyl]pyrimidine (100 mg, 0.477 mmol) (i.e. the product of Example 4, step A) in p-dioxane (3.5 mL, 0.136 M) in a microwave vial. Added palladium(II) acetate (6 mg), triphenyl phosphine (11 mg), copper(I) iodide (0.15 g), 1,8-diazabicyclo[5.4.0]undec-7-ene (0.12 g, 0.79 mmol) then capped. The reaction mixture was stirred a few minutes before placing in the microwave and heating at 140° C. for 2 h. An aliquat was removed from the reaction mixture for mass spectral analysis, which revealed the presence of the desired product material (Mass Spec. (dual) ES⁺=354, 1×Cl pattern). The reaction mixture was diluted with water and extracted (3×) with ethyl acetate. The combined organic layers were washed with brine, dried over MgSO₄, filtered and concentrated to provide an oil. The oil was purified using a 12 g MPLC column (silica gel) eluting with a gradient of 0 to 50% ethyl acetate in hexanes to isolate a the title compound as a solid (52 mg). Mass Spec (dual) ES⁺=354 (1×Cl pattern).

¹H NMR (500 MHz) δ ppm 2.45 (s, 3H), 5.55 (s, 2H), 7.73 (d, 2H), 7.92 (d, 2H), 8.71 (s, 2H).

¹⁹F NMR δ ppm −62.94 (s, 1×CF₃).

### Synthesis Example 5

### Preparation of 5-chloro-2-[[5-(methylsulfonyl)-3-(trifluoromethyl)-1H-1,2,4-triazol-1-yl]methyl]pyrimidine

### Step A: Preparation of 5-(methylthio)-3-(trifluoromethyl)-1H-1,2,4-triazole

Dissolved 5-(trifluoromethyl)-4H-1,2,4-triazole-3(2H)-thione (1 g, 5.9 mmol) in acetonitrile (25 mL) under a nitrogen atmosphere. Added potassium carbonate (2.21 g, 16.0 mmol) then iodomethane (0.834 g, 366 μL, 5.88 mmol) and stirred at ambient temperature for 16 h. The reaction mixture was diluted with water and extracted (3×) with ethyl acetate. The combined organic layers were washed with brine, dried over MgSO₄, filtered and concentrated to yield 0.57 g of an oil. The resulting oil was purified using a 12 g MPLC (silica gel) column eluting with a gradient of 0 to 20% ethyl acetate in hexanes to isolate the title compound as a white solid (0.33 g).

¹H NMR δ ppm 2.74 (s, 3H), 10.17-11.58 (bp, 1H).

¹⁹F NMR δ ppm −65.66 (br s, 1×CF₃).

### Step B: Preparation of 5-chloro-2-[[5-(methylthio)-3-(trifluoromethyl)-1H-1,2,4-triazol-1-yl]methyl]pyrimidine

Transferred 5-(methylthio)-3-(trifluoromethyl)-1H-1,2,4-triazole (0.28 g, 1.5 mmol, i.e. the product of Example 5, Step A) to a scintillation vial under a nitrogen atmosphere, with acetonitrile (6 mL). Potassium carbonate (0.634 g, 4.59 mmol) was added followed by 5-chloro-2-(chloromethyl)-pyrimidine hydrochloride (1:1) (0.366 g, 1.84 mmol) at ambient temperature then stirred for 16 h. The reaction mixture was diluted with water and extracted with ethyl acetate (3×). The combined organic layers were washed with water, brine, dried over MgSO₄, filtered, then concentrated to yield 0.51g oil. The oil was purified by silica gel chromatography using a 12 g MPLC column and eluting with 0 to 20% of an ethyl acetate in hexanes to isolate the title compound as an oil 0.31 g.

¹H NMR δ ppm 2.74 (s, 3H), 5.53 (s, 2H), 8.67 (s, 2H).

¹⁹F NMR δ ppm −65.53 (s, 1×CF₃).

### Step C: Preparation of 5-chloro-2-[[5-(methylsulfonyl)-3-(trifluoromethyl)-1H-1,2,4-triazol-1-yl]methyl]pyrimidine

Stirred 5-chloro-2-[[5-(methylthio)-3-(trifluoromethyl)-1H-1,2,4-triazol-1-yl]methyl]pyrimidine (0.31 g, 1.1 mmol, i.e. the product of Example 5, Step B) in dichloromethane (3 mL, 0.37 M) under a nitrogen atmosphere. Added 3-chloroperoxybenzoic acid 0.561 g, 2.44 mmol) at ambient temperature and stirred for 4 h. An aliquat was removed from the reaction mixture for mass spectral analysis, which revealed the presence of the desired product material along with the starting material (Mass Spec. (dual) ES⁺=354, 1×Cl pattern; organic layer (dual) ES⁺=342 and 326, 1×Cl pattern). The reaction mixture was diluted with saturated Na₂S₂O₅ aqueous solution, then extracted (3×) with dichloromethane. The combined organic layers were washed with brine, (and tested negative for peroxides with a test strip), dried over MgSO₄, filtered and concentrated to provide 0.64 g of an oil which solidified. Filtered off a white solid from hexanes to obtain the title compound as a solid (0.27 g).

¹H NMR δ ppm 3.24 (s, 3H, S(O)Me), 3.45 (s, 3H), 6.05 (s, 2H), 6.08 (s, 1H), 8.66 (m, 2H).

¹⁹F NMR δ ppm −65.24 (s, 1F) sulfoxide, −65.13 (s, 1×CF₃) sulfone. Indicated approximately 80% desired product and 20% sulfoxide intermediate.

### Synthesis Example 6

### Preparation of 5-chloro-2-[[3-(trifluoromethyl)-5-[3-(trifluoromethyl)-1H-pyrazol-1-yl]-1H-1,2,4-triazol-1-yl]methyl]pyrimidine (Compound 27)

### Step D: Preparation of 5-chloro-2-[[3-(trifluoromethyl)-5-[3-(trifluoromethyl)-1H-pyrazol-1-yl]-1H-1,2,4-triazol-1-yl]methyl]pyrimidine

Dissolved 3-(trifluoromethyl)-1H-pyrazole (20 mg, 0.147 mmol) in N,N-dimethylformamide (2 mL) under a nitrogen atmosphere. Added Cesium carbonate (0.072 g, 0.22 mmol) followed by 5-chloro-2-[[5-(methylsulfonyl)-3-(trifluoromethyl)-1H-1,2,4-triazol-1-yl]methyl]pyrimidine (0.075 g, 0.22 mmol, i.e. the product from Example 5, Step C). The reaction mixture was heated at 80° C. for 2 h, then allowed to stand at ambient temperature for 16 h. The reaction mixture was diluted with water then extracted with diethyl ether (3×). The combined organic layers were washed with water, dried over Na₂SO₄, filtered, then concentrated to provide an oil. The oil was purified by silica gel chromatography using a 12 g MPLC column eluting with 0 to 20% gradient of ethyl acetate in hexanes to isolate the title compound as a white solid 18.9 mg. Mass Spec (dual) ES⁺=398.3 and ES⁻=396 both (1×Cl) splitting pattern.

¹H NMR δ ppm 6.14 (s, 2H), 6.72 (d, 1H), 8.37-8.49 (m, 1H), 8.56 (s, 2H).

¹⁹F NMR δ ppm −65.88 (s, 1×CF₃), −63.06 (s, 1×CF₃).

### Synthesis Example 7

### Preparation of 5-chloro-2-[[5-(5-chloro-2-thienyl)-3-(trifluoromethyl)-1H-1,2,4-triazol-1-yl]methyl]pyrimidine

### Step A: Preparation of 2-[[5-bromo-3-(trifluoromethyl)-1H-1,2,4-triazol-1-yl]methyl]-5-chloropyrimidine

Dissolved 3-bromo-5-(trifluoromethyl)-2H-1,2,4-triazole (1.25 g, 5.79 mmol) in acetonitrile (20 mL, 0.29 M) under a nitrogen atmosphere. Potassium carbonate (2.4 g, 17 mmol) was added followed by 2-(bromomethyl)-5-chloropyrimidine (1.27 g, 6.12 mmol) and the mixture was allowed to stir ambient temperature for 16 h. The reaction mixture was diluted with water and extracted with ethyl acetate (3×). The combined organic layers were washed with water, brine, dried over MgSO₄, filtered and concentrated to yield 1.95 g of a solid. The solid was purified using a 12 g silica gel column) eluting with a gradient of 0 to 20% ethyl acetate in hexanes to isolate the title compound as an oil (1.44g) as a preferred common intermediate (i.e. to prepare a compound of Formula 1) which solidified upon standing. Mass Spec (dual) ES⁺=341.9 and ES⁻=339.9 (both with BrCl splitting pattern.

¹H NMR (500 MHz) δ ppm 5.67 (s, 2H), 8.67 (s, 2H).

¹⁹F NMR δ ppm −65.84 (s, 1×CF₃).

### Step B: Preparation of 5-chloro-2-[[5-(5-chloro-2-thienyl)-3-(trifluoromethyl)-1H-1,2,4-triazol-1-yl]methyl]pyrimidine

Combined 2-[[5-bromo-3-(trifluoromethyl)-1H-1,2,4-triazol-1-yl]methyl]-5-chloropyrimidine (i.e. the product obtained in Example 5, Step A) (0.1 g, 0.29 mmol) and 5-chlorothiophen-2-boronic acid (0.1 g, 0.614 mmol) in p-dioxanes (3 mL) under a nitrogen atmosphere. Added potassium phosphate tribasic (0.15 g, 0.71 mmol) and 1,1′-bis(diphenyl phosphino)ferrocene-palladium(II) dichloride dichloromethane complex (PdCl₂(dppf), 0.02 g, 0.024 mmol) then heated at 100° C. for 2 h. The reaction mixture was diluted with water then extracted with ethyl acetate (3×). The combined organic layers were washed with water, brine, dried over MgSO₄, filtered, then concentrated to yield 0.13g oil. The oil was purified by silica gel chromatography using a 12 g column eluting with 0 to 30% ethyl acetate in hexanes to isolate the compound as an oil (100 mg). Mass Spec (LCMS) ES⁺=380.0 (Cl₂) and ES⁻=377.9 (Cl₂).

¹H NMR δ ppm 5.77 (s, 2H), 6.94 (d, 1H), 7.36 (d, 1H), 8.69 (s, 2H).

¹⁹F NMR δ ppm −65.85 (s, 1×CF₃).

### Synthesis Example to Prepare an Intermediate

### Step A: Preparation of 5-chloro-2-[[5-iodo-3-(trifluoromethyl)-1H-1,2,4-triazol-1-yl]methyl]pyrimidine

Dissolved solid 5-Chloro-2-[[3-(trifluoromethyl)-1H-1,2,4-triazol-1-yl]methyl]pyrimidine (100 mg, 0.379 mmol) in 2 mL of tetrahydrofuran under a nitrogen atmosphere. The mixture was cooled to −5° C. with an ice-acetone bath. To the stirred mixture was added ZnTMPCl—LiCl solution (chloro(2,2,6,6-tetramethylpiperidin-1-yl) zinc, lithium chloride, 0.14 g, 0.49 mL, 1 M, 0.49 mmol) slowly via a syringe. The reaction mixture turned a light brown colored solution which was stirred at −5° C. for 15 min. Iodine (0.116 g, 0.455 mmol, 1.2 eq.) dissolved in 2 mL of tetrahydrofuran was added to the reaction mixture via a syringe and the mixture was allowed to warm to ambient temperature over 16 h. An aliquat was removed from the reaction mixture for mass spectral analysis, which revealed the presence of the desired product material and starting material. M.S. (dual) ES⁺=264 and 389.9 both (1×Cl) in 2:1 ratio). The reaction mixture was diluted with water, then extracted with ethyl acetate (3×). The combined organic layers were washed with saturated Na₂S₂O₅ aqueous solution, brine, dried over MgSO₄, filtered, and concentrated to provide 0.1 g of an oil. The oil was purified by silica gel column chromatography using a 12 g MPLC (silica gel) column eluting with 0 to 30% ethyl acetate in hexanes as a gradient to isolate the title compound, a preferred common intermediate (i.e. to prepare a compound of Formula 1) as a white solid (30 mg). Mass Spec (dual) ES⁺=390 (1×Cl pattern).

¹H NMR δ ppm 5.68 (s, 2H), 8.67 (s, 2H).

¹⁹F NMR δ ppm −65.59 (m, 1×CF₃).

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and 7-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically

C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 m can be wet milled using media mills to obtain particles with average diameters below 3 m. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 m range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Table A.

Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 1 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 1 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| Water | 20.0% |

### Example G

Suspension Concentrate

| | |
|---|---|
| Compound 1 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| Water | 53.7% |

### Example H

Emulsion in Water

| | |
|---|---|
| Compound 1 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| Water | 58.7% |

### Example I

Oil Dispersion

| | |
|---|---|
| Compound 1 | 25% |
| polyoxyethylene sorbitol hexaoleate | 15% |
| organically modified bentonite clay | 2.5% |
| fatty acid methyl ester | 57.5% |

Test results indicate that the compounds of the present invention are highly active preemergent and/or postemergent herbicides and/or plant growth regulants. The compounds of the invention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: Examples of additional active ingredients are allidochlor, acetochlor, acifluorfen and its sodium salt, aclonifen, acrolein (2-propenal), alachlor, alloxydim, ametryn, amicarbazone, amidosulfuron, aminocyclopyrachlor and its methyl and ethyl esters and its sodium and potassium salts, aminopyralid, amitrole, ammonium sulfamate, anilofos, asulam, atrazine, azimsulfuron, beflubutamid, beflubutamid-M, benazolin, benazolin-ethyl, bencarbazone, benfluralin, benfuresate, benquinotrione, bensulfuron-methyl, bensulide, bentazone, benzobicyclon, benzofenap, bicyclopyrone, bifenox, bilanafos, bipyrazone, bispyribac and its sodium salt, bixlozone, broclozone, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinflubrolin, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyranil, cyclopyrimorate, cyclosulfamuron, cycloxydim, cyhalofop-butyl, cypyrafluone, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimesulfazet, dimepiperate, dimesulfazet, dimethachlor, dimethametryn, dimethenamid, dimethenamid-P, dimethipin, dimethylarsinic acid and its sodium salt, dinitramine, dinoterb, dioxopyritrione, diphenamid, diquat dibromide, dithiopyr, diuron, DNOC, endothal, EPTC, epyrifenacil, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenpyrazone, fenquinotrione, fentrazamide, fenuron, fenuron-TCA, feproxydim, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, fluchloraminopyr, fluchloraminopyr-tefuryl, flufenacet, flufenoximacil, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, flusulfinam, fluthiacet-methyl, fomesafen, foramsulfuron, fosamine-ammonium, glufosinate, glufosinate-ammonium, L-glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, icafolin, icafolin-methyl, imazamethabenz-methyl, imazamox, imazapic, imazapyr, imazaquin, imazaquin-ammonium, imazethapyr, imazethapyr-ammonium, imazosulfuron, indanofan, indaziflam, indolauxipyr, indolauxipyr-cyanomethyl, iofensulfuron, iofensulfuron-sodium, iodosulfuron-methyl, iodosulfuron-sodium, ioxynil, ioxynil octanoate, ioxynil-sodium, ipfencarbazone, iptriazopyrid, isoproturon, isouron, isoxaben, isoxaflutole, isoxachlortole, lactofen, lancotrione, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metproxybicyclone, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyraquinate, pyrasulfotole, pyrazogyl, pyrazolynate, pyrazoxyfen, pyrazosulfuron-ethyl, pyribenzoxim, pyributicarb, pyridate, pyriflubenzoxim, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimisoxafen, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, tetflupyrolimet, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tripyrasulfone, tritosulfuron, vernolate, 3-(2-chloro-3,6-difluorophenyl)-4-hydroxy-1-methyl-1,5-naphthyridin-2(1H)-one, 6-chloro-4-(2,7-dimethyl-1-naphthalenyl)-5-hydroxy-2-methyl-3(2H)-pyridazinone, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide, 2-methyl-N-(4-methyl-1,2,5-oxadiazol-3-yl)-3-(methylsulfinyl)-4-(trifluoromethyl)benzamide, methyl 2-[2-[2-bromo-5-[3,6-dihydro-3-methyl-2,6-dioxo-4-(trifluoromethyl)-1(2H)-pyrimidinyl]-4-fluorophenoxy]phenoxy]-2-methoxyacetate, ethyl 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylate, 1-(2-carboxyethyl)-4-(2-pyrimidinyl)pyridazinium and its salts and esters. The compound of Formula 1 can also be mixed with a compound selected from the group consisting of
N-[2,4-dimethyl-5-(1-piperidinylcarbonyl)phenyl]-1,1,1-trifluoromethanesulfonamide;
N-[2-chloro-4-methyl-5-(4-morpholinylcarbonyl)phenyl]-1,1,1-trifluoromethanesulfonamide;
N-[2,4-dimethyl-5-(4-morpholinylcarbonyl)phenyl]-1,1,1-trifluoromethanesulfonamide;
N-[2-chloro-4-methyl-5-(1-piperidinylcarbonyl)phenyl]-1,1,1-trifluoromethanesulfonamide;
3-fluoro-N,N,2,4-tetramethyl-5-[[(trifluoromethyl)sulfonyl]amino]benzamide; 1,1,1-trifluoro-N-[3-fluoro-2,4-dimethyl-5-(4-morpholinylcarbonyl)phenyl]methanesulfonamide;
N-[2,4-dimethyl-5-(1-oxa-2-azaspiro[4.4]non-2-en-3-yl)phenyl]-1,1,1-trifluoromethanesulfonamide;
N-[2,4-dimethyl-5-[(3aR,6aR)-3a,5,6,6a-tetrahydro-4H-cyclopent[d]isoxazol-3-yl]phenyl]-1,1,1-trifluoromethanesulfonamide;
N-[2,4-dimethyl-5-(1-oxa-2-azaspiro[4.5]dec-2-en-3-yl)phenyl]-1,1,1-trifluoromethanesulfonamide;
N-[2,4-dimethyl-5-(3a,4,7,7a-tetrahydro-5H-pyrano[4,3-d]isoxazol-3-yl)phenyl]-1,1,1-trifluoromethanesulfonamide;
N-[2,4-dimethyl-5-(3a,6,7,7a-tetrahydro-4H-pyrano[3,4-d]isoxazol-3-yl)phenyl]-1,1,1-trifluoromethanesulfonamide;
N-[2,4-dimethyl-5-(1-oxo-2-azaspiro[4.5]dec-2-yl)phenyl]-1,1,1-trifluoro-N-[(trifluoromethyl)sulfonyl]methanesulfonamide;
[[2,4-dimethyl-5-(1-oxa-2-azaspiro[4.4]non-2-en-3-yl)phenyl][(trifluoromethyl)sulfonyl]amino]methyl 2,2-dimethylpropanoate;
[[2,4-dimethyl-5-(1-oxa-2-azaspiro[4.5]dec-2-en-3-yl)phenyl][(trifluoromethyl)sulfonyl]amino]methyl 2,2-dimethylpropanoate;
[[2,4-dimethyl-5-[(3aR,6aR)-3a,5,6,6a-tetrahydro-4H-cyclopent[d]isoxazol-3-yl]phenyl][(trifluoromethyl)sulfonyl]amino]methyl 2,2-dimethylpropanoate;
[[2,4-dimethyl-5-(1-oxo-2-azaspiro[4.5]dec-2-yl)phenyl][(trifluoromethyl)sulfonyl]amino]methyl 2,2-dimethylpropanoate;
[[(trifluoromethyl)sulfonyl][2,3,4-trimethyl-5-(4-morpholinylcarbonyl)phenyl]amino]methyl 2,2-dimethylpropanoate; ethyl N-[(trifluoromethyl)sulfonyl]-N-[2,3,4-trimethyl-5-(1-piperidinylcarbonyl)phenyl]carbamate;
[[(trifluoromethyl)sulfonyl][2,3,4-trimethyl-5-(1-piperidinylcarbonyl)phenyl]amino]methyl 2,2-dimethylpropanoate; 1,1,1-trifluoro-N-[2,3,4-trimethyl-5-(4-morpholinylcarbonyl)phenyl]methanesulfonamide; and
[[(trifluoromethyl)sulfonyl][2,3,4-trimethyl-5-[(3aR,6aR)-3a,5,6,6a-tetrahydro-4H-cyclopent[d]isoxazol-3-yl]phenyl]amino]methyl 2,2-dimethylpropanoate;

The compound of Formula 1 can also be mixed with a compound selected from the group consisting of
[(2S)-8-Chloro-2,3-dihydro-2-methyl-4H-1,4-benzoxazin-4-yl][2-methyl-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-[3-(1,1-dimethylethyl)-1H-1,2,4-triazol-1-yl]-2-methylphenyl]methanone;
[(2S)-8-Chloro-2,3-dihydro-2-methyl-4H-1,4-benzoxazin-4-yl][2-methoxy-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-(3-ethyl-1H-1,2,4-triazol-1-yl)-2-methoxyphenyl]methanone;
[(2S)-8-Chloro-2,3-dihydro-2-methyl-4H-1,4-benzoxazin-4-yl][2-ethyl-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-(3-cyclobutyl-1H-1,2,4-triazol-1-yl)-2-methylphenyl]methanone;
[(2S)-8-Chloro-2,3-dihydro-2-methyl-4H-1,4-benzoxazin-4-yl][3-(3-cyclobutyl-1H-1,2,4-triazol-1-yl)phenyl]methanone;
[(2S)-2,3-Dihydro-2,8-dimethyl-4H-1,4-benzoxazin-4-yl][2-methoxy-5-(1H-1,2,4-triazol-1-yl)phenyl]methanone;

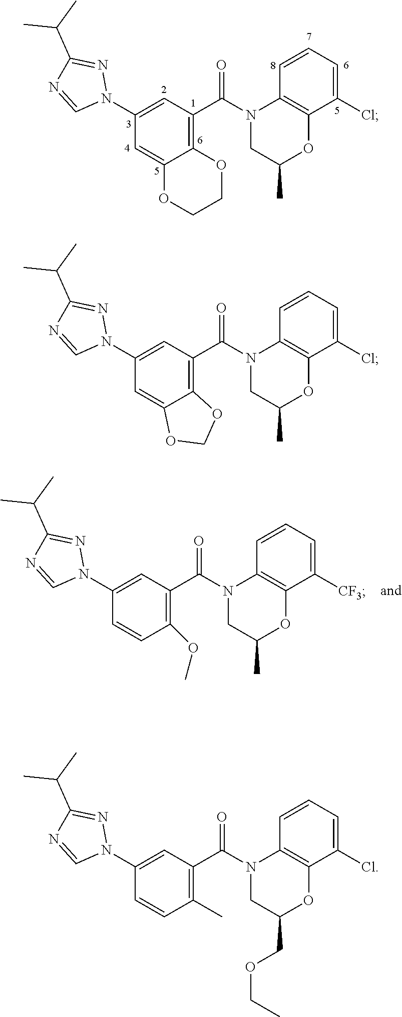 [SMILES: CC(C)c1ncn(-c2cc3c(c(C(=O)N4C[C@H](C)Oc5c(Cl)cccc54)c2)OCCO3)n1 ; CC(C)c1ncn(-c2cc3c(c(C(=O)N4C[C@H](C)Oc5c(Cl)cccc54)c2)OCO3)n1 ; COc1ccc(-n2cnc(C(C)C)n2)cc1C(=O)N1C[C@H](C)Oc2c1cccc2C(F)(F)F ; CCOC[C@H]1CN(C(=O)c2cc(-n3cnc(C(C)C)n3)ccc2C)c2cccc(Cl)c2O1]

The compound of Formula 1 can also be mixed with a compound selected from the group consisting of
2,2,2-trifluoroethyl (4S)-4-[[[(5S)-3-(3,5-difluorophenyl)-5-methyl-2-oxo-5-oxazolidinyl]carbonyl]amino]-1-cyclopentene-1-carboxylate;
2,2,2-trifluoroethyl (4S)-4-[[[(5R)-3-(3,5-difluorophenyl)-5-methyl-2-oxo-5-oxazolidinyl]carbonyl]amino]-1-cyclopentene-1-carboxylate;
2-(methylthio)ethyl (4S)-4-[[[(5S)-3-(3,5-difluorophenyl)-5-methyl-2-oxo-5-oxazolidinyl]carbonyl]amino]-1-cyclopentene-1-carboxylate;
2-(methylthio)ethyl (4S)-4-[[[(5R)-3-(3,5-difluorophenyl)-5-methyl-2-oxo-5-oxazolidinyl]carbonyl]amino]-1-cyclopentene-1-carboxylate;
methyl (4S)-4-[[[(5S)-3-(3,5-difluorophenyl)-5-methyl-2-oxo-5-oxazolidinyl]carbonyl]amino]-1-cyclopentene-1-carboxylate;
methyl (4S)-4-[[[(5R)-3-(3,5-difluorophenyl)-5-methyl-2-oxo-5-oxazolidinyl]carbonyl]amino]-1-cyclopentene-1-carboxylate;
methyl (4S)-4-[[[3-(3,5-difluorophenyl)-5-methyl-2-oxo-5-oxazolidinyl]carbonyl]amino]-1-cyclopentene-1-carboxylate;
1-methylethyl (1S,4R)-4-[[[3-(3-chloro-5-fluorophenyl)-5-methyl-2-oxo-5-oxazolidinyl]carbonyl]amino]-2-cyclopentene-1-carboxylate;
methyl (1S,4R)-4-[[[3-(3-chloro-5-fluorophenyl)-5-methyl-2-oxo-5-oxazolidinyl]carbonyl]amino]-2-cyclopentene-1-carboxylate;
2-methylpropyl (4S)-4-[[[3-(3,5-difluorophenyl)-5-methyl-2-oxo-5-oxazolidinyl]carbonyl]amino]-1-cyclopentene-1-carboxylate;
(1S,4R)-4-[[[3-(3,5-difluorophenyl)-5-methyl-2-oxo-5-oxazolidinyl]carbonyl]amino]-2-cyclopentene-1-carboxylic acid;
(4S)-4-[[[3-(3,5-difluorophenyl)-5-methyl-2-oxo-5-oxazolidinyl]carbonyl]amino]-1-cyclopentene-1-carboxylic acid;
(1S,4R)-4-[[[3-(3-chloro-5-fluorophenyl)-5-methyl-2-oxo-5-oxazolidinyl]carbonyl]amino]-2-cyclopentene-1-carboxylic acid; and
ethyl (1S,4R)-4-[[[3-(3,5-difluorophenyl)-5-methyl-2-oxo-5-oxazolidinyl]carbonyl]amino]-2-cyclopentene-1-carboxylate.

The compound of Formula 1 can also be mixed with a compound selected from the group consisting of
4-[(E)-(3-bromo-1-naphthalenyl)(methoxyimino)methyl]-5-hydroxy-2,6-dimethyl-3(2H)-pyridazinone;
4-[(Z)-(3-bromo-1-naphthalenyl)(methoxyimino)methyl]-5-hydroxy-2,6-dimethyl-3(2H)-pyridazinone;
4-[(E)-(3-bromo-1-naphthalenyl)[(2-propyn-1-yloxy)imino]methyl]-5-hydroxy-2,6-dimethyl-3(2H)-pyridazinone;
4-[(E)-(3-bromo-1-naphthalenyl)(ethoxyimino)methyl]-5-hydroxy-2,6-dimethyl-3(2H)-pyridazinone;
4-[(Z)-(4-fluoro-1-naphthalenyl)[(2-propyn-1-yloxy)imino]methyl]-5-hydroxy-2,6-dimethyl-3(2H)-pyridazinone; and
4-[(E)-(4-fluoro-1-naphthalenyl)[(2-propyn-1-yloxy)imino]methyl]-5-hydroxy-2,6-dimethyl-3(2H)-pyridazinone.

The compound of Formula 1 can also be mixed with a compound selected from the group consisting of
4-[[2-(4-fluorophenyl)-5-methyl-2H-1,2,3-triazol-4-yl]methyl]-2-(trifluoromethyl)pyridine;
4-[[2-(4-fluorophenyl)-5-methyl-2H-1,2,3-triazol-4-yl]oxy]-2-(trifluoromethyl)pyridine;
4-[[5-ethoxy-2-(4-fluorophenyl)-2H-1,2,3-triazol-4-yl]methyl]-2-(trifluoromethyl)pyridine;
4-[[5-methoxy-2-[4-(trifluoromethyl)phenyl]-2H-1,2,3-triazol-4-yl]methyl]-2-(trifluoromethyl)pyridine;
4-[[5-methyl-2-[4-(trifluoromethyl)phenyl]-2H-1,2,3-triazol-4-yl]methyl]-2-(trifluoromethyl)pyridine;
4-[[5-ethoxy-2-[4-(trifluoromethyl)phenyl]-2H-1,2,3-triazol-4-yl]methyl]-2-(trifluoromethyl)pyridine;
4-[[5-(2,2,2-trifluoroethoxy)-2-[4-(trifluoromethyl)phenyl]-2H-1,2,3-triazol-4-yl]methyl]-2-(trifluoromethyl)pyridine;
4-[[5-ethyl-3-[4-(trifluoromethyl)phenyl]-1H-1,2,4-triazol-1-yl]methyl]-2-(trifluoromethyl)pyridine;
4-[[3-(4-fluorophenyl)-5-propyl-1H-1,2,4-triazol-1-yl]methyl]-2-(trifluoromethyl)pyridine;
4-[[5-ethoxy-3-(4-fluorophenyl)-1H-1,2,4-triazol-1-yl]methyl]-2-(trifluoromethyl)pyridine;
4-[[3-(4-fluorophenyl)-1-propyl-1H-1,2,4-triazol-5-yl]methyl]-2-(trifluoromethyl)pyridine; and
4-[[3-(4-fluorophenyl)-5-methoxy-1H-1,2,4-triazol-1-yl]methyl]-2-(trifluoromethyl)pyridine.

Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub, their environmentally compatible salts, “acids”, esters and amides, and combinations thereof.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners (e.g., such as those listed in (b18)) to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention cans also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Preferred for better control of undesired vegetation (e.g., lower use rate such as from synergism, broader spectrum of weeds controlled, or enhanced crop safety) or for preventing the development of resistant weeds are mixtures of a compound of this invention with a second herbicide Table A1 lists specific combinations of a Component (a) with Component (b) illustrative of the mixtures, compositions and methods of the present invention. Compound 1 in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 1 in Index Table A) with 2,4-D is typically applied in a weight ratio between 1:192-6:1. The remaining lines of Table A1 are to be construed similarly.

TABLE A1

| Component (a) | | Typical | More Typical | Most Typical |
|---|---|---|---|---|
| (Compound 1) | Component (b) | Weight Ratio | Weight Ratio | Weight Ratio |
| 1 | 2,4-D | 1:192-6:1 | 1:64-2:1 | 1:24-1:3 |
| 1 | Acetochlor | 1:768-2:1 | 1:256-1:2 | 1:96-1:11 |
| 1 | Acifluorfen | 1:96-12:1 | 1:32-4:1 | 1:12-1:2 |
| 1 | Aclonifen | 1:857-2:1 | 1:285-1:3 | 1:107-1:12 |
| 1 | Alachlor | 1:768-2:1 | 1:256-1:2 | 1:96-1:11 |
| 1 | Ametryn | 1:384-3:1 | 1:128-1:1 | 1:48-1:6 |
| 1 | Amicarbazone | 1:192-6:1 | 1:64-2:1 | 1:24-1:3 |
| 1 | Amidosulfuron | 1:6-168:1 | 1:2-56:1 | 1:1-11:1 |
| 1 | Aminocyclopyrachlor | 1:48-24:1 | 1:16-8:1 | 1:6-2:1 |
| 1 | Aminopyralid | 1:20-56:1 | 1:6-19:1 | 1:2-4:1 |
| 1 | Amitrole | 1:768-2:1 | 1:256-1:2 | 1:96-1:11 |
| 1 | Anilofos | 1:96-12:1 | 1:32-4:1 | 1:12-1:2 |
| 1 | Asulam | 1:960-2:1 | 1:320-1:3 | 1:120-1:14 |
| 1 | Atrazine | 1:192-6:1 | 1:64-2:1 | 1:24-1:3 |
| 1 | Azimsulfuron | 1:6-168:1 | 1:2-56:1 | 1:1-11:1 |
| 1 | Beflubutamid | 1:342-4:1 | 1:114-2:1 | 1:42-1:5 |
| 1 | Benfuresate | 1:617-2:1 | 1:205-1:2 | 1:77-1:9 |
| 1 | Bensulfuron-methyl | 1:25-45:1 | 1:8-15:1 | 1:3-3:1 |
| 1 | Bentazone | 1:192-6:1 | 1:64-2:1 | 1:24-1:3 |
| 1 | Benzobicyclon | 1:85-14:1 | 1:28-5:1 | 1:10-1:2 |
| 1 | Benzofenap | 1:257-5:1 | 1:85-2:1 | 1:32-1:4 |
| 1 | Bicyclopyrone | 1:42-27:1 | 1:14-9:1 | 1:5-2:1 |
| 1 | Bifenox | 1:257-5:1 | 1:85-2:1 | 1:32-1:4 |
| 1 | Bispyribac-sodium | 1:10-112:1 | 1:3-38:1 | 1:1-7:1 |
| 1 | Bromacil | 1:384-3:1 | 1:128-1:1 | 1:48-1:6 |
| 1 | Bromobutide | 1:384-3:1 | 1:128-1:1 | 1:48-1:6 |
| 1 | Bromoxynil | 1:96-12:1 | 1:32-4:1 | 1:12-1:2 |
| 1 | Butachlor | 1:768-2:1 | 1:256-1:2 | 1:96-1:11 |
| 1 | Butafenacil | 1:42-27:1 | 1:14-9:1 | 1:5-2:1 |
| 1 | Butylate | 1:1542-1:2 | 1:514-1:5 | 1:192-1:22 |
| 1 | Carfenstrole | 1:192-6:1 | 1:64-2:1 | 1:24-1:3 |
| 1 | Carfentrazone-ethyl | 1:128-9:1 | 1:42-3:1 | 1:16-1:2 |
| 1 | Chlorimuron-ethyl | 1:8-135:1 | 1:2-45:1 | 1:1-9:1 |
| 1 | Chlorotoluron | 1:768-2:1 | 1:256-1:2 | 1:96-1:11 |
| 1 | Chlorsulfuron | 1:6-168:1 | 1:2-56:1 | 1:1-11:1 |
| 1 | Cincosulfuron | 1:17-68:1 | 1:5-23:1 | 1:2-5:1 |
| 1 | Cinidon-ethyl | 1:384-3:1 | 1:128-1:1 | 1:48-1:6 |
| 1 | Cinmethylin | 1:34-34:1 | 1:11-12:1 | 1:4-3:1 |
| 1 | Clacyfos | 1:34-34:1 | 1:11-12:1 | 1:4-3:1 |
| 1 | Clethodim | 1:48-24:1 | 1:16-8:1 | 1:6-2:1 |
| 1 | Clodinafop-propargyl | 1:20-56:1 | 1:6-19:1 | 1:2-4:1 |
| 1 | Clomazone | 1:384-3:1 | 1:128-1:1 | 1:48-1:6 |
| 1 | Clomeprop | 1:171-7:1 | 1:57-3:1 | 1:21-1:3 |
| 1 | Clopyralid | 1:192-6:1 | 1:64-2:1 | 1:24-1:3 |
| 1 | Cloransulam-methyl | 1:12-96:1 | 1:4-32:1 | 1:1-6:1 |
| 1 | Cumyluron | 1:384-3:1 | 1:128-1:1 | 1:48-1:6 |
| 1 | Cyanazine | 1:384-3:1 | 1:128-1:1 | 1:48-1:6 |
| 1 | Cyclopyrimorate | 1:17-68:1 | 1:5-23:1 | 1:2-5:1 |
| 1 | Cyclosulfamuron | 1:17-68:1 | 1:5-23:1 | 1:2-5:1 |
| 1 | Cycloxydim | 1:96-12:1 | 1:32-4:1 | 1:12-1:2 |
| 1 | Cyhalofop | 1:25-45:1 | 1:8-15:1 | 1:3-3:1 |
| 1 | Daimuron | 1:192-6:1 | 1:64-2:1 | 1:24-1:3 |
| 1 | Desmedipham | 1:322-4:1 | 1:107-2:1 | 1:40-1:5 |
| 1 | Dicamba | 1:192-6:1 | 1:64-2:1 | 1:24-1:3 |
| 1 | Dichlobenil | 1:1371-1:2 | 1:457-1:4 | 1:171-1:20 |
| 1 | Dichlorprop | 1:925-2:1 | 1:308-1:3 | 1:115-1:13 |
| 1 | Diclofop-methyl | 1:384-3:1 | 1:128-1:1 | 1:48-1:6 |
| 1 | Diclosulam | 1:10-112:1 | 1:3-38:1 | 1:1-7:1 |
| 1 | Difenzoquat | 1:288-4:1 | 1:96-2:1 | 1:36-1:4 |
| 1 | Diflufenican | 1:857-2:1 | 1:285-1:3 | 1:107-1:12 |
| 1 | Diflufenzopyr | 1:12-96:1 | 1:4-32:1 | 1:1-6:1 |
| 1 | Dimethachlor | 1:768-2:1 | 1:256-1:2 | 1:96-1:11 |
| 1 | Dimethametryn | 1:192-6:1 | 1:64-2:1 | 1:24-1:3 |
| 1 | Dimethenamid-P | 1:384-3:1 | 1:128-1:1 | 1:48-1:6 |
| 1 | Dithiopyr | 1:192-6:1 | 1:64-2:1 | 1:24-1:3 |
| 1 | Diuron | 1:384-3:1 | 1:128-1:1 | 1:48-1:6 |
| 1 | EPTC | 1:768-2:1 | 1:256-1:2 | 1:96-1:11 |
| 1 | Esprocarb | 1:1371-1:2 | 1:457-1:4 | 1:171-1:20 |
| 1 | Ethalfluralin | 1:384-3:1 | 1:128-1:1 | 1:48-1:6 |
| 1 | Ethametsulfuron-methyl | 1:17-68:1 | 1:5-23:1 | 1:2-5:1 |
| 1 | Ethoxyfen | 1:8-135:1 | 1:2-45:1 | 1:1-9:1 |
| 1 | Ethoxysulfuron | 1:20-56:1 | 1:6-19:1 | 1:2-4:1 |
| 1 | Etobenzanid | 1:257-5:1 | 1:85-2:1 | 1:32-1:4 |
| 1 | Fenoxaprop-ethyl | 1:120-10:1 | 1:40-4:1 | 1:15-1:2 |
| 1 | Fenoxasulfone | 1:85-14:1 | 1:28-5:1 | 1:10-1:2 |
| 1 | Fenquinotrione | 1:17-68:1 | 1:5-23:1 | 1:2-5:1 |
| 1 | Fentrazamide | 1:17-68:1 | 1:5-23:1 | 1:2-5:1 |
| 1 | Flazasulfuron | 1:17-68:1 | 1:5-23:1 | 1:2-5:1 |
| 1 | Florasulam | 1:2-420:1 | 1:1-140:1 | 2:1-27:1 |
| 1 | Fluazifop-butyl | 1:192-6:1 | 1:64-2:1 | 1:24-1:3 |
| 1 | Flucarbazone | 1:8-135:1 | 1:2-45:1 | 1:1-9:1 |
| 1 | Flucetosulfuron | 1:8-135:1 | 1:2-45:1 | 1:1-9:1 |
| 1 | Flufenacet | 1:257-5:1 | 1:85-2:1 | 1:32-1:4 |
| 1 | Flumetsulam | 1:24-48:1 | 1:8-16:1 | 1:3-3:1 |
| 1 | Flumiclorac-pentyl | 1:10-112:1 | 1:3-38:1 | 1:1-7:1 |
| 1 | Flumioxazin | 1:25-45:1 | 1:8-15:1 | 1:3-3:1 |
| 1 | Fluometuron | 1:384-3:1 | 1:128-1:1 | 1:48-1:6 |
| 1 | Flupyrsulfuron-methyl | 1:3-336:1 | 1:1-112:1 | 2:1-21:1 |
| 1 | Fluridone | 1:384-3:1 | 1:128-1:1 | 1:48-1:6 |
| 1 | Fluroxypyr | 1:96-12:1 | 1:32-4:1 | 1:12-1:2 |
| 1 | Flurtamone | 1:857-2:1 | 1:285-1:3 | 1:107-1:12 |
| 1 | Fluthiacet-methyl | 1:48-42:1 | 1:16-14:1 | 1:3-3:1 |
| 1 | Fomesafen | 1:96-12:1 | 1:32-4:1 | 1:12-1:2 |
| 1 | Foramsulfuron | 1:13-84:1 | 1:4-28:1 | 1:1-6:1 |
| 1 | Glufosinate | 1:288-4:1 | 1:96-2:1 | 1:36-1:4 |
| 1 | Glyphosate | 1:288-4:1 | 1:96-2:1 | 1:36-1:4 |
| 1 | Halosulfuron-methyl | 1:17-68:1 | 1:5-23:1 | 1:2-5:1 |
| 1 | Halauxifen | 1:20-56:1 | 1:6-19:1 | 1:2-4:1 |
| 1 | Halauxifen methyl | 1:20-56:1 | 1:6-19:1 | 1:2-4:1 |
| 1 | Haloxyfop-methyl | 1:34-34:1 | 1:11-12:1 | 1:4-3:1 |
| 1 | Hexazinone | 1:192-6:1 | 1:64-2:1 | 1:24-1:3 |
| 1 | Hydantocidin | 1:1100-16:1 | 1:385-8:1 | 1:144-4:1 |
| 1 | Imazamox | 1:13-84:1 | 1:4-28:1 | 1:1-6:1 |
| 1 | Imazapic | 1:20-56:1 | 1:6-19:1 | 1:2-4:1 |
| 1 | Imazapyr | 1:85-14:1 | 1:28-5:1 | 1:10-1:2 |
| 1 | Imazaquin | 1:34-34:1 | 1:11-12:1 | 1:4-3:1 |
| 1 | Imazethabenz-methyl | 1:171-7:1 | 1:57-3:1 | 1:21-1:3 |
| 1 | Imazethapyr | 1:24-48:1 | 1:8-16:1 | 1:3-3:1 |
| 1 | Imazosulfuron | 1:27-42:1 | 1:9-14:1 | 1:3-3:1 |
| 1 | Indanofan | 1:342-4:1 | 1:114-2:1 | 1:42-1:5 |
| 1 | Indaziflam | 1:25-45:1 | 1:8-15:1 | 1:3-3:1 |
| 1 | Iodosulfuron-methyl | 1:3-336:1 | 1:1-112:1 | 2:1-21:1 |
| 1 | Ioxynil | 1:192-6:1 | 1:64-2:1 | 1:24-1:3 |
| 1 | Ipfencarbazone | 1:85-14:1 | 1:28-5:1 | 1:10-1:2 |
| 1 | Isoproturon | 1:384-3:1 | 1:128-1:1 | 1:48-1:6 |
| 1 | Isoxaben | 1:288-4:1 | 1:96-2:1 | 1:36-1:4 |
| 1 | Isoxaflutole | 1:60-20:1 | 1:20-7:1 | 1:7-2:1 |
| 1 | Lactofen | 1:42-27:1 | 1:14-9:1 | 1:5-2:1 |
| 1 | Lenacil | 1:384-3:1 | 1:128-1:1 | 1:48-1:6 |
| 1 | Linuron | 1:384-3:1 | 1:128-1:1 | 1:48-1:6 |
| 1 | MCPA | 1:192-6:1 | 1:64-2:1 | 1:24-1:3 |
| 1 | MCPB | 1:288-4:1 | 1:96-2:1 | 1:36-1:4 |
| 1 | Mecoprop | 1:768-2:1 | 1:256-1:2 | 1:96-1:11 |
| 1 | Mefenacet | 1:384-3:1 | 1:128-1:1 | 1:48-1:6 |
| 1 | Mefluidide | 1:192-6:1 | 1:64-2:1 | 1:24-1:3 |
| 1 | Mesosulfuron-methyl | 1:5-224:1 | 1:1-75:1 | 1:1-14:1 |
| 1 | Mesotrione | 1:42-27:1 | 1:14-9:1 | 1:5-2:1 |
| 1 | Metamifop | 1:42-27:1 | 1:14-9:1 | 1:5-2:1 |
| 1 | Metazachlor | 1:384-3:1 | 1:128-1:1 | 1:48-1:6 |
| 1 | Metazosulfuron | 1:25-45:1 | 1:8-15:1 | 1:3-3:1 |
| 1 | Methabenzthiazuron | 1:768-2:1 | 1:256-1:2 | 1:96-1:11 |
| 1 | Metolachlor | 1:768-2:1 | 1:256-1:2 | 1:96-1:11 |
| 1 | Metosulam | 1:8-135:1 | 1:2-45:1 | 1:1-9:1 |
| 1 | Metribuzin | 1:192-6:1 | 1:64-2:1 | 1:24-1:3 |
| 1 | Metsulfuron-methyl | 1:2-560:1 | 1:1-187:1 | 3:1-35:1 |
| 1 | Molinate | 1:1028-2:1 | 1:342-1:3 | 1:128-1:15 |
| 1 | Napropamide | 1:384-3:1 | 1:128-1:1 | 1:48-1:6 |
| 1 | Napropamide-M | 1:192-6:1 | 1:64-2:1 | 1:24-1:3 |
| 1 | Naptalam | 1:192-6:1 | 1:64-2:1 | 1:24-1:3 |
| 1 | Nicosulfuron | 1:12-96:1 | 1:4-32:1 | 1:1-6:1 |
| 1 | Norflurazon | 1:1152-1:1 | 1:384-1:3 | 1:144-1:16 |
| 1 | Orbencarb | 1:1371-1:2 | 1:457-1:4 | 1:171-1:20 |
| 1 | Orthosulfamuron | 1:20-56:1 | 1:6-19:1 | 1:2-4:1 |
| 1 | Oryzalin | 1:514-3:1 | 1:171-1:2 | 1:64-1:8 |
| 1 | Oxadiargyl | 1:384-3:1 | 1:128-1:1 | 1:48-1:6 |
| 1 | Oxadiazon | 1:548-3:1 | 1:182-1:2 | 1:68-1:8 |
| 1 | Oxasulfuron | 1:27-42:1 | 1:9-14:1 | 1:3-3:1 |
| 1 | Oxaziclomefone | 1:42-27:1 | 1:14-9:1 | 1:5-2:1 |
| 1 | Oxyfluorfen | 1:384-3:1 | 1:128-1:1 | 1:48-1:6 |
| 1 | Paraquat | 1:192-6:1 | 1:64-2:1 | 1:24-1:3 |
| 1 | Pendimethalin | 1:384-3:1 | 1:128-1:1 | 1:48-1:6 |
| 1 | Penoxsulam | 1:10-112:1 | 1:3-38:1 | 1:1-7:1 |
| 1 | Penthoxamid | 1:384-3:1 | 1:128-1:1 | 1:48-1:6 |
| 1 | Pentoxazone | 1:102-12:1 | 1:34-4:1 | 1:12-1:2 |
| 1 | Phenmedipham | 1:102-12:1 | 1:34-4:1 | 1:12-1:2 |
| 1 | Picloram | 1:96-12:1 | 1:32-4:1 | 1:12-1:2 |
| 1 | Picolinafen | 1:34-34:1 | 1:11-12:1 | 1:4-3:1 |
| 1 | Pinoxaden | 1:25-45:1 | 1:8-15:1 | 1:3-3:1 |
| 1 | Pretilachlor | 1:192-6:1 | 1:64-2:1 | 1:24-1:3 |
| 1 | Primisulfuron-methyl | 1:8-135:1 | 1:2-45:1 | 1:1-9:1 |
| 1 | Prodiamine | 1:384-3:1 | 1:128-1:1 | 1:48-1:6 |
| 1 | Profoxydim | 1:42-27:1 | 1:14-9:1 | 1:5-2:1 |
| 1 | Prometryn | 1:384-3:1 | 1:128-1:1 | 1:48-1:6 |
| 1 | Propachlor | 1:1152-1:1 | 1:384-1:3 | 1:144-1:16 |
| 1 | Propanil | 1:384-3:1 | 1:128-1:1 | 1:48-1:6 |
| 1 | Propaquizafop | 1:48-24:1 | 1:16-8:1 | 1:6-2:1 |
| 1 | Propoxycarbazone | 1:17-68:1 | 1:5-23:1 | 1:2-5:1 |
| 1 | Propyrisulfuron | 1:17-68:1 | 1:5-23:1 | 1:2-5:1 |
| 1 | Propyzamide | 1:384-3:1 | 1:128-1:1 | 1:48-1:6 |
| 1 | Prosulfocarb | 1:1200-1:2 | 1:400-1:4 | 1:150-1:17 |
| 1 | Prosulfuron | 1:6-168:1 | 1:2-56:1 | 1:1-11:1 |
| 1 | Pyraclonil | 1:42-27:1 | 1:14-9:1 | 1:5-2:1 |
| 1 | Pyraflufen-ethyl | 1:5-224:1 | 1:1-75:1 | 1:1-14:1 |
| 1 | Pyrasulfotole | 1:13-84:1 | 1:4-28:1 | 1:1-6:1 |
| 1 | Pyrazolynate | 1:857-2:1 | 1:285-1:3 | 1:107-1:12 |
| 1 | Pyrazosulfuron-ethyl | 1:10-112:1 | 1:3-38:1 | 1:1-7:1 |
| 1 | Pyrazoxyfen | 1:5-224:1 | 1:1-75:1 | 1:1-14:1 |
| 1 | Pyribenzoxim | 1:10-112:1 | 1:3-38:1 | 1:1-7:1 |
| 1 | Pyributicarb | 1:384-3:1 | 1:128-1:1 | 1:48-1:6 |
| 1 | Pyridate | 1:288-4:1 | 1:96-2:1 | 1:36-1:4 |
| 1 | Pyriftalid | 1:10-112:1 | 1:3-38:1 | 1:1-7:1 |
| 1 | Pyriminobac-methyl | 1:20-56:1 | 1:6-19:1 | 1:2-4:1 |
| 1 | Pyrimisulfan | 1:17-68:1 | 1:5-23:1 | 1:2-5:1 |
| 1 | Pyrithiobac | 1:24-48:1 | 1:8-16:1 | 1:3-3:1 |
| 1 | Pyroxasulfone | 1:85-14:1 | 1:28-5:1 | 1:10-1:2 |
| 1 | Pyroxsulam | 1:5-224:1 | 1:1-75:1 | 1:1-14:1 |
| 1 | Quinclorac | 1:192-6:1 | 1:64-2:1 | 1:24-1:3 |
| 1 | Quizalofop-ethyl | 1:42-27:1 | 1:14-9:1 | 1:5-2:1 |
| 1 | Rimsulfuron | 1:13-84:1 | 1:4-28:1 | 1:1-6:1 |
| 1 | Saflufenacil | 1:25-45:1 | 1:8-15:1 | 1:3-3:1 |
| 1 | Sethoxydim | 1:96-12:1 | 1:32-4:1 | 1:12-1:2 |
| 1 | Simazine | 1:384-3:1 | 1:128-1:1 | 1:48-1:6 |
| 1 | Sulcotrione | 1:120-10:1 | 1:40-4:1 | 1:15-1:2 |
| 1 | Sulfentrazone | 1:147-8:1 | 1:49-3:1 | 1:18-1:3 |
| 1 | Sulfometuron-methyl | 1:34-34:1 | 1:11-12:1 | 1:4-3:1 |
| 1 | Sulfosulfuron | 1:8-135:1 | 1:2-45:1 | 1:1-9:1 |
| 1 | Tebuthiuron | 1:384-3:1 | 1:128-1:1 | 1:48-1:6 |
| 1 | Tefuryltrione | 1:42-27:1 | 1:14-9:1 | 1:5-2:1 |
| 1 | Tembotrione | 1:31-37:1 | 1:10-13:1 | 1:3-3:1 |
| 1 | Tepraloxydim | 1:25-45:1 | 1:8-15:1 | 1:3-3:1 |
| 1 | Terbacil | 1:288-4:1 | 1:96-2:1 | 1:36-1:4 |
| 1 | Terbuthylazine | 1:857-2:1 | 1:285-1:3 | 1:107-1:12 |
| 1 | Terbutryn | 1:192-6:1 | 1:64-2:1 | 1:24-1:3 |
| 1 | Thenylchlor | 1:85-14:1 | 1:28-5:1 | 1:10-1:2 |
| 1 | Thiazopyr | 1:384-3:1 | 1:128-1:1 | 1:48-1:6 |
| 1 | Thiencarbazone | 1:3-336:1 | 1:1-112:1 | 2:1-21:1 |
| 1 | Thifensulfuron-methyl | 1:5-224:1 | 1:1-75:1 | 1:1-14:1 |
| 1 | Tiafenacil | 1:17-68:1 | 1:5-23:1 | 1:2-5:1 |
| 1 | Thiobencarb | 1:768-2:1 | 1:256-1:2 | 1:96-1:11 |
| 1 | Tolpyralate | 1:31-37:1 | 1:10-13:1 | 1:3-3:1 |
| 1 | Topramzone | 1:6-168:1 | 1:2-56:1 | 1:1-11:1 |
| 1 | Tralkoxydim | 1:68-17:1 | 1:22-6:1 | 1:8-2:1 |
| 1 | Triafamone | 1:2-420:1 | 1:1-140:1 | 2:1-27:1 |
| 1 | Triallate | 1:768-2:1 | 1:256-1:2 | 1:96-1:11 |
| 1 | Triasulfuron | 1:5-224:1 | 1:1-75:1 | 1:1-14:1 |
| 1 | Triaziflam | 1:171-7:1 | 1:57-3:1 | 1:21-1:3 |
| 1 | Tribenuron-methyl | 1:3-336:1 | 1:1-112:1 | 2:1-21:1 |
| 1 | Triclopyr | 1:192-6:1 | 1:64-2:1 | 1:24-1:3 |
| 1 | Trifloxysulfuron | 1:2-420:1 | 1:1-140:1 | 2:1-27:1 |
| 1 | Trifludimoxazin | 1:25-45:1 | 1:8-15:1 | 1:3-3:1 |
| 1 | Trifluralin | 1:288-4:1 | 1:96-2:1 | 1:36-1:4 |
| 1 | Triflusulfuron-methyl | 1:17-68:1 | 1:5-23:1 | 1:2-5:1 |
| 1 | Tritosulfuron | 1:13-84:1 | 1:4-28:1 | 1:1-6:1 |

Table A2 is constructed the same as Table A1 above except that entries below the “Component (a)” column heading are replaced with the respective Component (a) Column Entry shown below. Compound 1 in the Component (a) column is identified in Index Table A. Thus, for example, in Table A2 the entries below the “Component (a)” column heading all recite “Compound 2” (i.e. Compound 2 identified in Index Table A), and the first line below the column headings in Table A2 specifically discloses a mixture of Compound 2 with 2,4-D. Tables A3 through A47 are constructed similarly.

| Table Number | Component (a) Column Entries |
|---|---|
| A2 | Compound 2 |
| A3 | Compound 3 |
| A4 | Compound 4 |
| A5 | Compound 5 |
| A6 | Compound 6 |
| A7 | Compound 7 |
| A8 | Compound 8 |
| A9 | Compound 9 |
| A10 | Compound10 |
| A11 | Compound 11 |
| A12 | Compound 12 |
| A13 | Compound 13 |
| A14 | Compound 14 |
| A15 | Compound 15 |
| A16 | Compound 16 |
| A17 | Compound 17 |
| A18 | Compound 18 |
| A19 | Compound 19 |
| A20 | Compound 20 |
| A21 | Compound 21 |
| A22 | Compound 22 |
| A23 | Compound 23 |
| A24 | Compound 24 |
| A25 | Compound 25 |
| A26 | Compound 26 |
| A27 | Compound 27 |
| A28 | Compound 28 |
| A29 | Compound 29 |
| A30 | Compound 30 |
| A31 | Compound 31 |
| A32 | Compound 32 |
| A33 | Compound 33 |
| A34 | Compound 34 |
| A35 | Compound 35 |
| A36 | Compound 36 |
| A37 | Compound 37 |
| A38 | Compound 38 |
| A39 | Compound 39 |
| A40 | Compound 40 |
| A41 | Compound 41 |
| A42 | Compound 42 |
| A43 | Compound 43 |
| A44 | Compound 44 |
| A45 | Compound 45 |
| A46 | Compound 46 |
| A47 | Compound 47 |

Preferred for better control of undesired vegetation (e.g., lower use rate such as from synergism, broader spectrum of weeds controlled, or enhanced crop safety) or for preventing the development of resistant weeds are mixtures of a compound of this invention with a herbicide selected from the group consisting of bixlozone, clomazone, beflubutamid-M, chlorimuron-ethyl, nicosulfuron, mesotrione, thifensulfuron-methyl, flupyrsulfuron-methyl, tribenuron, pyroxasulfone, pinoxaden, rimisoxafen, tembotrione, pyroxsulam, tetflupyrolimet, metolachlor and S-metolachlor.

The following Tests demonstrate the control efficacy of the compounds of this invention against specific weeds. The weed control afforded by the compounds is not limited, however, to these species. See Index Tables A for compound descriptions. The following abbreviations are used in the Index Tables which follow: t is tertiary, s is secondary, n is normal, i is iso, c is cyclo, Me is methyl, Et is ethyl, Pr is propyl, i-Pr is isopropyl, Bu is butyl, c-Pr is cyclopropyl, t-Bu is tert-butyl, Ph is phenyl, OMe is methoxy, OEt is ethoxy, SMe is methylthio, SEt is ethylthio, —CN is cyano, —NO₂ is nitro, TMS is trimethylsilyl, and naphthyl means naphthalenyl. The abbreviation “Cmpd. No.” stands for “Compound Number”. The abbreviation “Ex.” stands for “Example” and is followed by a number indicating in which example the compound is prepared. Mass spectra are reported with an estimated precision within ±0.5 Da as the molecular weight of the highest isotopic abundance parent ion (M+1) formed by addition of H+(molecular weight of 1) to the molecule observed by using atmospheric pressure chemical ionization (AP+).

INDEX TABLE A

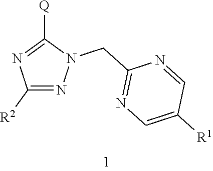

| Cmpd. No. | R¹ | R² | R³ | Q | m.p. (° C.) |
|---|---|---|---|---|---|
| 1 (Ex.1) | Cl | CF₃ | CF₃ | G-1 | * |
| 2 (Ex. 2) | Cl | CF₃ | Cl | G-1 | * |
| 3 (Ex. 4) | Cl | CH₃ | CF₃ | G-1 | * |
| 4 (Ex. 3) | Cl | CF₃ | CF₃ | G-6 | * |
| 5 | Cl | CF₃ | i-Pr | G-16 | * |
| 6 | Cl | CF₃ | Et | G-16 | * |
| 7 (Ex. 7) | Cl | CF₃ | Cl | G-22 | * |
| 8 | Cl | CF₃ | CHF₂ | G-16 | * |
| 9 | Cl | CF₃ | CH₃ | G-18 | * |
| 10 | Cl | CF₃ | F, F | G-2 | * |
| 11 | Cl | CF₃ | Cl, Cl | G-2 | * |
| 12 | Cl | CF₃ | Cl, F | G-2 | * |
| 13 | Cl | CF₃ | F, Cl | G-2 | * |
| 14 | Cl | CF₃ | Cl | G-21 | * |
| 15 | Cl | CF₃ | CH₃, CH₃ | G-17 | * |
| 16 | Cl | CF₃ | CF₃, CH₃ | G-17 | * |
| 17 | Cl | CF₃ | CF₃ | G-21 | * |
| 18 | Cl | CF₃ | CN | G-1 | * |
| 19 | Cl | CF₃ | F | G-1 | * |
| 20 (Ex. 5) | Cl | CF₃ | — | SO₂CH₃ | * |
| 21 | Cl | CF₃ | — | SCH₂CH₂CH₂CF₃ | * |
| 22 | Cl | CF₃ | — | SCH₂CH₂CF₃ | * |
| 23 | Cl | CF₃ | — | OCH₂CH₂CH₂CF_{C} | * |
| 24 | Cl | CF₃ | CH₃, F | G-2 | * |
| 25 | Cl | CF₃ | CF₃ | G-20 | * |
| 26 | Cl | CF₃ | Br | G-20 | * |
| 27 (Ex. 6) | Cl | CF₃ | CF₃ | G-19 | * |
| 28 | Cl | CF₃ | Cl | G-6 | * |
| 29 | Cl | CF₃ | F | G-6 | * |
| 30 | Cl | CHF₂ | CF₃ | G-1 | * |
| 31 | Cl | CF₃ | — | n-Pr | * |
| 32 | Cl | CF₃ | F,F | G-5 | * |
| 33 | Cl | CF₃ | CN | G-4 | * |
| 34 | Cl | CF₃ | CH₃ | G-8 | * |
| 35 | Cl | CF₃ | Cl, CH₃ | G-7 | * |
| 36 | Cl | CF₃ | F, CH₃ | G-7 | * |
| 37 | Cl | CF₃ | Cl, F | G-7 | * |
| 38 | Cl | CF₃ | Cl, Cl | G-7 | * |
| 39 | Cl | CF₃ | CN | G-6 | * |
| 40 | Cl | CF₃ | CF₃ | G-4 | * |
| 41 | Cl | CHF₂ | Cl | G-1 | * |
| 42 | Cl | CF₃ | Cl | OCH₂CF₃ | * |
| 43 | Cl | CF₃ | F | G-4 | * |
| 44 | Cl | CF₃ | F, F | G-7 | * |
| 45 | Cl | CF₃ | Cl | G-4 | * |
| 46 | Cl | CF₃ | CH₃ | G-6 | * |
| 47 | Cl | CF₃ | F, F, F | G-23 | * |

* See Index Table B for ¹H NMR data.

INDEX TABLE B

| Cmpd. | ¹H NMR Data (400 MHz in CDCl₃, solution unless indicated otherwise indicated, δ ppm)^{a} or Mass |
|---|---|
| No | Spec data. ¹⁹F NMR at 471 MHz, in CDCl₃ δ ppm downfield from CFCl₃) |
| 1 | 500 MHz; 5.68 (s, 2 H), 7.77 (d, 2 H), 7.95 (br d, 2 H), 8.70 (s, 2 H). 19F δ ppm −65.30 (s, 1 × |
| | CF₃), −63.09 (s, 1 × CF₃). |
| 2 | 500 MHz; 5.66 (s, 2H), 7.44-7.55 (m, 2H), 7.66-7.86 (m, 2H), 8.59-8.79 (m, 2H). 19F δ ppm −65.32 |
| | (s, 1 × CF₃). |
| 3 | 500 MHz; 2.45 (s, 3H), 5.55 (s, 2H), 7.73 (d, 2H), 7.92 (d, 2H), 8.71 (s, 2H). 19F δ ppm −62.94 |
| | (s, 1 × CF₃). |
| 4 | 500 MHz; 5.70 (s, 2H), 7.86 (dd, 1H), 8.42 (dd, 1H), 8.71 (s, 2H), 9.20 (d, 1H). 19F δ ppm −68.23 |
| | (s, 1 × CF₃), −65.33 (s, 1 × CF₃). |
| 5 | 500 MHz; 1.53 (s, 3H), 1.54 (s, 3H), 4.54 (dt, 1H), 5.72 (s, 2H), 7.79 (d, 1H), 8.08 (s, 1H), 8.69 |
| | (s, 2H). 19F δ ppm −65.36 (s, 1 × CF₃). |
| 6 | 500 MHz; 1.52 (t, 3H, Me), 4.23 (q, 2H, N—CH₂Me), 5.72 (s, 2H, N—CH₂), 7.81 (s, 1H), 8.03 (s, 1H), |
| | 8.69 (s, 2H, pyrmidine H's). 19F δ ppm −65.37 (s, 1 × CF₃). |
| 7 | 500 MHz; 5.77 (s, 2H), 6.94 (d, 1H), 7.36 (d, 1H), 8.69 (s, 2H). 19F δ ppm −65.85 (s, 1 × CF₃). |
| 8 | 500 MHz; 5.71 (s, 2H, N—CH₂), 7.24 (t, 1H, CHF2), 8.09 (d, 1H, pyrz-H), 8.47 (s, 1H, pyrz-H), 8.72 |
| | (s, 2H, pyrmidine H's). 19F δ ppm −94.26 (d, 1 × CHF2), −65.40 (s, 1 × CF₃). |
| 9 | 500 MHz; 4.14 (s, 3H), 5.72 (s, 2H), 6.58 (d, 1H), 7.53 (d, 1H), 8.67 (s, 2H). 19F δ ppm −65.34 |
| | (s, 1 × CF₃). |
| 10 | 5.66 (s, 2H), 7.31 (m, 1H), 7.57 (m, 1H), 7.39 (m, 1H), 8.71 (s, 2H). |
| 11 | 5.66 (s, 2H), 7.57 (d, 1H), 7.65 (m, 1H), 7.98 (d, 1H), 8.71 (s, 2H). |
| 12 | 5.66 (s, 2H), 7.26 (m, 1H), 7.70 (m, 1H), 7.95 (dd, 1H), 8.71 (s, 2H). |
| 13 | 5.67 (s, 2H), 7.55 (m, 2H), 7.70 (dd, 1H), 8.71 (s, 2H). |
| 14 | 5.67 (s, 2H), 7.42 (t, 1H), 7.52 (d, 1H), 7.65 (d, 1H), 7.83 (s, 1H), 8.70 (s, 2H). |
| 15 | 500 MHz; 2.27 (s, 3H, c-Me), 4.06 (s, 3H, N—Me), 5.71 (s, 2H, N—CH₂), 6.32 (s, 1H), 8.67 (s, 2H, |
| | pyrimidine H's). 19F δ ppm −65.34 (s, 1 × CF₃). |
| 16 | 500 MHz; 4.19 (s, 3H, N—Me), 5.71 (s, 2H, N—CH₂), 6.93 (s, 1H), 8.69 (s, 2H, pyrmidine H's). 19F δ |
| | ppm −65.36and −62.11 each (s, 1 × CF₃). |
| 17 | 5.67 (s, 2H), 7.64 (t, 1H), 7.80 (d, 1H), 8.0 (d, 1H), 8.14 (s, 1H), 8.71 (s, 2H). |
| 18 | 5.68 (s, 2H), 7.79 (d, 2H), 7.97 (d, 2H), 8.70 (s, 2H). |
| 19 | 5.65 (s, 2H), 7.18 (t, 2H), 7.91 (m, 2H), 8.70 (s, 2H). |
| 20 | 500 MHz; 3.24 (s, 1H, S(O)Me), 3.45 (s, 3H, S(O)2Me), 6.05 (s, 2H, N—CH₂), 6.08 (s, 1H), 8.66 (m, |
| | 2H, pyrmidine H's). 19F δ ppm −65.24 (s, 1 F), sulfoxide, −65.13 (s, 1 × CF₃), sulfone. |
| 21 | 500 MHz; 1.99-2.10 (m, 2H), 2.18-2.32 (m, 2H), 3.35 (t, 2H), 5.54 (s, 2H), 8.66 (s, 2H). 19F δ |
| | ppm −65.98 (t, 1 × CF₃), −65.57 (s, 1 × CF₃). |
| 22 | 500 MHz; 2.48-2.84 (m, 2H), 3.27-3.58 (m, 2H), 5.55 (s, 2H), 8.66 (s, 2H). 19F δ ppm −66.02 (t, 1 × |
| | CF₃), −65.57 (s, 1 × CF₃). |
| 23 | 500 MHz; 2.02-2.11 (m, 2H), 2.14-2.27 (m, 2H), 4.57 (br t, 2H), 5.41 (s, 2H), 8.65 (s, 2H). 19F δ |
| | ppm −66.44 (s, 1 × CF₃), −66.35 (t, 1 × CF₃). |
| 24 | 2.33 (s, 3H), 5.67 (s, 2H), 7.30 (m, 1H), 7.47 (m, 2H), 8.70 (s, 2H). |
| 25 | 500 MHz; 6.17 (s, 2H), 7.80 (s, 1H), 8.58 (s, 2H), 8.69 (s, 1H). 19F δ ppm −65.94 (s, 1 × CF₃), −57.48 |
| | (s, 1 × CF₃). |
| 26 | 500 MHz; 6.14 (s, 2H), 7.56 (d, 1H), 8.39 (d, 1H), 8.57 (s, 2H). 19F δ ppm −65.93 (s, 1 × CF₃). |
| 27 | 500 MHz 6.14 (s, 2H) 6.72 (d, 1H) 8.37-8.49 (m, 1H) 8.56 (s, 2H). 19F δ ppm −65.88 (s, 1 × |
| | CF₃), −63.06 (s, 1 × CF₃). |
| 28 | 5.67 (s, 2H), 7.50 (d, 1H), 8.16 (dd, 1H), 8.69 (s, 2H), 8.85 (m, H). |
| 29 | 5.67 (s, 2H), 7.11 (dd, 1H), 8.30 (m, 1H), 8.70 (s, 3H). |
| 30 | 500 MHz; 5.65 (s, 2H), 6.75 (t, 1H), 7.77 (m, 2H), 7.95 (d, 2H), 8.70 (s, 2H). 19F δ ppm −117.09 (s, 1 |
| | CHF), −116.97 (s, 1 CHF), −63.06 (s, 1 × CF₃). |
| 31 | 500 MHz; 1.00 (t, 3H), 1.74-1.95 (m, 2H), 2.63-2.93 (m, 2H), 5.56 (s, 2H, N—CH₂), 8.66 (s, 2H, |
| | pyrmidine H's). 19F δ ppm −65.34 (s, 1 × CF₃). |
| 32 | 500 MHz; 5.73 (s, 2H, N—CH₂), 7.39 (s, 2H), 8.72 (s, 2H, pyrmidine H's). |
| 33 | 500 MHz; 5.72 (s, 2H), 8.02 (dd, 1H), 8.27 (dd, 1H), 8.73 (s, 2H), 8.90 (dd, 1H). 19F δ ppm −65.37 (s, |
| | 1 × CF₃). |
| 34 | 2.83 (s, 3H), 5.67 (s, 2H), 8.69 (d, 2H), 9.10 (d, 2H). |
| 35 | 2.46 (s, 3H), 5.67 (s, 2H), 8.069 (d, 1H), 8.650 (d, 1H), 8.69 (s, 2H). |
| 36 | 2.36 (s, 3H), 5.67 (s, 2H), 8.11 (dd, 1H), 8.46 (d, 1H), 8.699 (s, 2H). |
| 37 | 5.68 (s, 2H), 8.09 (dd, 1H), 8.70 (m, 3H). |
| 38 | 5.68 (s, 2H), 8.36 (d, 1H), 8.71 (s, 2H), 8.77 (d, 1H). |
| 39 | 5.70 (s, 2H), 7.86 (d, 1H), 8.41 (dd, 1H), 8.70 (d, 2H), 9.21 (d, 1H). |
| 40 | 500 MHz; 5.72 (s, 2H), 7.98 (dd, 1H), 8.25 (s, 1H), 8.72 (s, 2H), 8.91 (d, 1H). 19F δ ppm −68.19 (s, |
| | 1 × CF₃), −65.34 (s, 1 × CF₃). |
| 41 | 500 MHz; 5.63 (s, 2H), 6.73 (t, 1H), 7.47 (d, 2H), 7.74 (d, 2H), 8.70 (s, 2H). 19F NMR (471 MHz, |
| | CDCL3-d) δ ppm −117.06 (s, 1 × CHF), −116.94 (s, 1 × CHF). |
| 42 | 500 MHz; 4.87 (q, 2H, OCH₂), 5.46 (s, 2H, N—CH₂), 8.65 (s, 2H, pyrmidine H's). 19F δ ppm −74.39 |
| | (t, 1 × CF₃), −66.49 (s, 1 × CF₃). |
| 43 | 500 MHz; 5.67 (s, 2H), 7.11 (dd, 1H), 8.30 (ddd, 1H), 8.70 (s, 3H). |
| 44 | 5.68 (s, 2H), 8.19 (m, 1H), 8.48 (t, 1H), 8.71 (s, 2H). |
| 45 | 500 MHz; 5.71 (s, 2H), 7.68 (dd, 1H), 7.86 (d, 1H), 8.56 (dd, 1H), 8.71 (s, 2H). 19F δ ppm −65.34 (s, |
| | 1 × CF₃). |
| 46 | 2.637 (s, 3H), 5.67 (s, 2H), 7.31 (d, 1H), 8.03 (dd, 1H), 8.69 (s, 2H), 8.88 (d, 1H). |
| 47 | 500 MHz; 5.66 (s, 2H), 7.58 (dd, 2H), 8.72 (s, 2H). 19F δ ppm −154.82 (br dd, 1 Ar—F), −131.06 (dd, |
| | 2 Ar—F), −65.40 (s, 1 × CF₃). |

^{a1}H NMR data are in ppm downfield from tetramethylsilane.
b* = 1H NMR (500 MHz, CDCL3-d).
Couplings are designated by (s)—singlet, (d)—doublet, (t)—triplet, (m)—multiplet, (dd)—doublet of doublets, (dt)—doublet of triplets, ddd—doublet of double doublets, (br s)—broad singlet, (br p)—broad peak.

### BIOLOGICAL EXAMPLES OF THE INVENTION

### Test A

Seeds of plant species selected from barnyardgrass (Echinochloa crusgalli), blackgrass (Alopecurus myosuroides), corn (Zea mays), green foxtail (Setaria viridis), kochia (Bassia scoparia), wild oat (Avena fatua), palmer amaranth (palmer pigweed, Amaranthus palmeri), ragweed (common ragweed, Ambrosia artemisiifolia), Italian ryegrass (Lolium multiflorum), soybean (Glycine max), and wheat (Triticum aestivum) were planted into a blend of loam soil and sand and treated preemergence with a directed soil spray using test chemicals formulated in a non-phytotoxic solvent mixture which included a surfactant.

At the same time, plants selected from these crop and weed species and also galium (catchweed bedstraw, Galium aparine) and horseweed (Erigeron canadensis) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for 10 d, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| 1000 g ai/ha | Compound |
|---|---|
| Preemergence | 20 |
| Blackgrass | 0 |
| Foxtail, Green | 10 |
| Kochia | 80 |
| Pigweed, Palmer | 60 |
| Ragweed | 20 |
| Ryegrass, Italian | 0 |

| 500 g ai/ha | Compounds | | | | | | |
|---|---|---|---|---|---|---|---|
| Preemergence | 5 | 6 | 7 | 8 | 9 | 26 | 27 |
| Barnyardgrass | 100 | 30 | 100 | 100 | 100 | — | — |
| Blackgrass | 90 | 30 | 90 | 90 | 90 | 0 | 70 |
| Corn | 60 | 0 | 40 | 50 | 50 | 0 | 10 |
| Foxtail, Green | 100 | 100 | 100 | 100 | 100 | 0 | 100 |
| Kochia | 100 | 90 | 100 | 100 | 100 | 0 | 90 |
| Oat, Wild | 90 | 0 | 80 | 60 | 30 | 0 | 10 |
| Pigweed, Palmer | 100 | 100 | 100 | 100 | 100 | 50 | 100 |
| Ragweed | 90 | 70 | 90 | 100 | 100 | 0 | 0 |
| Ryegrass, Italian | 100 | 30 | 100 | 100 | 60 | 0 | 20 |
| Soybean | 90 | 50 | 40 | 90 | 90 | 0 | 0 |
| Wheat | 10 | 0 | 70 | 30 | 20 | 0 | 0 |

| 125 g ai/ha | Compounds | | | | | | | | | | | | | |
|---|---|---|---|---|---|---|---|---|---|---|---|---|---|---|
| Preemergence | 1 | 2 | 3 | 4 | 5 | 6 | 7 | 8 | 9 | 10 | 11 | 12 | 13 | 14 |
| Barnyardgrass | 100 | 100 | 0 | 100 | 10 | 0 | 90 | 30 | 50 | 100 | 100 | 100 | 100 | 80 |
| Blackgrass | 90 | 90 | 10 | 90 | 30 | 0 | 90 | 50 | 20 | 90 | 70 | 90 | 90 | 90 |
| Corn | 50 | 30 | 0 | 60 | 10 | 0 | 10 | 10 | 10 | 70 | 10 | 20 | 20 | 0 |
| Foxtail, Green | 100 | 100 | 30 | 100 | 100 | 30 | 100 | 100 | 100 | 100 | 100 | 100 | 100 | 100 |
| Kochia | 100 | 100 | 70 | 100 | 90 | 20 | 100 | 100 | 100 | 100 | 100 | 100 | 100 | 90 |
| Oat, Wild | 90 | 40 | 0 | 100 | 0 | 0 | 10 | 10 | 0 | 70 | 20 | 50 | 70 | 10 |
| Pigweed, Palmer | 100 | 100 | 90 | 100 | 90 | 80 | 100 | 100 | 100 | — | — | — | — | — |
| Ragweed | 60 | 20 | 0 | 90 | 10 | 0 | 70 | 90 | 70 | 100 | 30 | 70 | 40 | 70 |
| Ryegrass, Italian | 100 | 80 | 0 | 90 | 20 | 0 | 90 | 60 | 20 | 100 | 100 | 100 | 100 | 90 |
| Soybean | 70 | 60 | 0 | 90 | 80 | 20 | 20 | 80 | 60 | 90 | 0 | 40 | 60 | 50 |
| Wheat | 30 | 10 | 0 | 80 | 0 | 0 | 0 | 0 | 0 | 40 | 10 | 10 | 40 | 0 |

| 125 g ai/ha | Compounds | | | | | | | | | | | |
|---|---|---|---|---|---|---|---|---|---|---|---|---|
| Preemergence | 15 | 16 | 17 | 18 | 19 | 21 | 22 | 23 | 24 | 25 | 26 | 27 |
| Barnyardgrass | 0 | 20 | 100 | 90 | 90 | 20 | 0 | 0 | 90 | 0 | — | — |
| Blackgrass | 10 | 20 | 90 | 70 | 70 | 0 | 0 | 0 | 90 | 0 | 0 | 0 |
| Corn | 0 | 0 | 30 | 70 | 60 | 0 | 0 | 0 | 40 | 0 | 0 | 0 |
| Foxtail, Green | 70 | 90 | 100 | 100 | 100 | 0 | 0 | 0 | 100 | 0 | 0 | 30 |
| Kochia | 80 | 60 | 100 | 100 | 100 | 0 | 80 | 0 | 100 | 0 | 0 | 0 |
| Oat, Wild | 0 | 0 | 80 | 40 | 20 | 0 | 0 | 0 | 40 | 0 | 0 | 0 |
| Pigweed, Palmer | — | — | — | 100 | 100 | 0 | 0 | 0 | 100 | 20 | 0 | 70 |
| Ragweed | 0 | 0 | 90 | 100 | 70 | 0 | 0 | 0 | 70 | 0 | 0 | 0 |
| Ryegrass, Italian | 10 | 20 | 100 | 90 | 50 | 0 | 0 | 0 | 90 | 0 | 0 | 0 |
| Soybean | 20 | 0 | 90 | 80 | 90 | 30 | 0 | 0 | 90 | 0 | 0 | 0 |
| Wheat | 0 | 0 | 50 | 40 | 10 | 0 | 0 | 0 | 30 | 0 | 0 | 0 |

| 31 g ai/ha | Compounds | | | | | | | | | | | | | |
|---|---|---|---|---|---|---|---|---|---|---|---|---|---|---|
| Preemergence | 1 | 2 | 3 | 4 | 10 | 11 | 12 | 13 | 14 | 15 | 16 | 17 | 18 | 19 |
| Barnyardgrass | 90 | 70 | 0 | 80 | 60 | 20 | 70 | 60 | 20 | 0 | 0 | 70 | 10 | 30 |
| Blackgrass | 90 | 20 | 0 | 60 | 50 | 50 | 60 | 50 | 10 | 0 | 0 | 90 | 10 | 50 |
| Corn | 10 | 0 | 0 | 30 | 10 | 0 | 0 | 0 | 0 | 0 | 0 | 10 | 20 | 0 |
| Foxtail, Green | 100 | 100 | 0 | 90 | 100 | 100 | 100 | 100 | 90 | 10 | 10 | 100 | 100 | 90 |
| Kochia | 100 | 100 | 0 | 100 | 100 | 80 | 90 | 90 | 90 | 10 | 20 | 90 | 100 | 100 |
| Oat, Wild | 20 | 10 | 0 | 30 | 10 | 0 | 0 | 10 | 0 | 0 | 0 | 10 | 0 | 10 |
| Pigweed, Palmer | 100 | 100 | 30 | 90 | — | — | — | — | — | — | — | — | 100 | 100 |
| Ragweed | 0 | 0 | 0 | 30 | 30 | 0 | 0 | 20 | 10 | 0 | 0 | 20 | 30 | 0 |
| Ryegrass, Italian | 90 | 30 | 0 | 80 | 40 | 60 | 70 | 60 | 10 | 0 | 0 | 70 | 10 | 10 |
| Soybean | 20 | 10 | 0 | 50 | 10 | 0 | 0 | 50 | 0 | 0 | 0 | 50 | 10 | 50 |
| Wheat | 10 | 0 | 0 | 0 | 0 | 0 | 0 | 10 | 0 | 0 | 0 | 0 | 0 | 0 |

| | |
|---|---|
| 31 g ai/ha | Compounds |

| Preemergence | 21 | 22 | 23 | 24 | 25 |
|---|---|---|---|---|---|
| Barnyardgrass | 10 | 0 | 0 | 50 | 0 |
| Blackgrass | 0 | 0 | 0 | 20 | 0 |
| Corn | 0 | 0 | 0 | 20 | 0 |
| Foxtail, Green | 0 | 0 | 0 | 90 | 0 |
| Kochia | 0 | 0 | 0 | 90 | 0 |
| Oat, Wild | 0 | 0 | 0 | 10 | 0 |
| Pigweed, Palmer | 0 | 0 | 0 | 90 | 0 |
| Ragweed | 0 | 0 | 0 | 10 | 0 |
| Ryegrass, Italian | 0 | 0 | 0 | 40 | 0 |
| Soybean | 0 | 0 | 0 | 10 | 0 |
| Wheat | 0 | 0 | 0 | 0 | 0 |

| 1000 g ai/ha | Compound |
|---|---|
| Postemergence | 20 |
| Barnyardgrass | 0 |
| Blackgrass | 10 |
| Foxtail, Green | 0 |
| Galium | 10 |
| Horseweed | 0 |
| Kochia | 30 |
| Pigweed, Palmer | 10 |
| Ragweed | 20 |
| Ryegrass, Italian | 0 |

| | |
|---|---|
| 500 g ai/ha | Compounds |

| Postemergence | 5 | 6 | 7 | 8 | 9 | 26 | 27 |
|---|---|---|---|---|---|---|---|
| Barnyardgrass | 70 | 30 | 80 | 80 | 50 | 0 | 30 |
| Blackgrass | 60 | 10 | 90 | 80 | 30 | 0 | 30 |
| Corn | 50 | 0 | 70 | 50 | 30 | 0 | 10 |
| Foxtail, Green | 80 | 10 | 90 | 90 | 90 | 0 | 30 |
| Galium | 90 | 60 | 90 | 90 | 90 | 0 | 10 |
| Horseweed | 20 | 20 | 50 | 50 | 20 | 0 | 10 |
| Kochia | 90 | 80 | 90 | 90 | 90 | 20 | 40 |
| Oat, Wild | 50 | 0 | 100 | 30 | 10 | 0 | 10 |
| Pigweed, Palmer | 80 | 80 | 100 | 100 | 100 | 30 | 70 |
| Ragweed | 80 | 30 | 80 | 80 | 80 | 0 | 10 |
| Ryegrass, Italian | 80 | 10 | 100 | 70 | 10 | 0 | 10 |
| Soybean | 100 | 80 | 90 | 90 | 90 | 10 | 30 |
| Wheat | — | — | — | — | — | 0 | 10 |

| 125 g ai/ha | Compounds | | | | | | | | | | | | | |
|---|---|---|---|---|---|---|---|---|---|---|---|---|---|---|
| Postemergence | 1 | 2 | 3 | 4 | 5 | 6 | 7 | 8 | 9 | 10 | 11 | 12 | 13 | 14 |
| Barnyardgrass | 90 | 90 | 40 | 100 | 10 | 20 | 70 | 50 | 10 | 90 | 70 | 90 | 80 | 40 |
| Blackgrass | 90 | 80 | 10 | 90 | 0 | 0 | 80 | 20 | 0 | 80 | 70 | 90 | 80 | 60 |
| Corn | 70 | 50 | 10 | 80 | 10 | 0 | 20 | 20 | 0 | 60 | 30 | 30 | 30 | 20 |
| Foxtail, Green | 90 | 90 | 0 | 90 | 10 | 0 | 90 | 40 | 10 | — | — | — | — | — |
| Galium | 100 | 100 | 70 | 100 | 50 | 20 | 90 | 80 | 50 | 90 | 90 | 90 | 90 | 80 |
| Horseweed | 80 | 100 | 10 | 50 | 20 | 10 | 30 | 30 | 20 | — | — | — | — | — |
| Kochia | 90 | 90 | 30 | 90 | 60 | 10 | 90 | 90 | 90 | 90 | 90 | 90 | 90 | 90 |
| Oat, Wild | 80 | 40 | 0 | 50 | 0 | 0 | 30 | 10 | 0 | 60 | 30 | 50 | 60 | 30 |
| Pigweed, Palmer | 100 | 100 | 60 | 100 | 60 | 20 | 100 | 100 | 90 | 100 | 100 | 100 | 100 | 100 |
| Ragweed | 80 | 70 | 20 | 80 | 30 | 20 | 40 | 70 | 30 | 70 | 60 | 40 | 50 | 20 |
| Ryegrass, Italian | 100 | 80 | 0 | 90 | 0 | 0 | 70 | 10 | 10 | 90 | 70 | 90 | 90 | 40 |
| Soybean | 90 | 70 | 50 | 100 | 90 | 30 | 60 | 80 | 70 | 100 | 90 | 80 | 80 | 80 |
| Wheat | 30 | 10 | 0 | 30 | — | — | — | — | — | 40 | 20 | 20 | 30 | 10 |

| | |
|---|---|
| 125 g ai/ha | Compounds |

| Postemergence | 15 | 16 | 17 | 18 | 19 | 21 | 22 | 23 | 24 | 25 | 26 | 27 |
|---|---|---|---|---|---|---|---|---|---|---|---|---|
| Barnyardgrass | 30 | 30 | 80 | 90 | 80 | 0 | 0 | 0 | 80 | 0 | 0 | 0 |
| Blackgrass | 0 | 10 | 90 | 80 | 80 | 0 | 0 | 0 | 80 | 0 | 0 | 0 |
| Corn | 10 | 10 | 40 | 70 | 40 | 0 | 0 | 0 | 60 | 0 | 0 | 0 |
| Foxtail, Green | — | — | — | 80 | 60 | 0 | 0 | 0 | 80 | 0 | 0 | 0 |
| Galium | 30 | 60 | 90 | 90 | 90 | 0 | 50 | 0 | 90 | 0 | 0 | 10 |
| Horseweed | — | — | — | 70 | 30 | 0 | 0 | 0 | 30 | 0 | 0 | 0 |
| Kochia | 70 | 40 | 100 | 90 | 90 | 0 | 10 | 0 | 90 | 10 | 0 | 10 |
| Oat, Wild | 0 | 10 | 40 | 40 | 40 | 0 | 0 | 0 | 50 | 0 | 0 | 0 |
| Pigweed, Palmer | 30 | 60 | 100 | 100 | 90 | 10 | 10 | 10 | 80 | 10 | 10 | 30 |
| Ragweed | 10 | 20 | 40 | 90 | 60 | 0 | 0 | 0 | 30 | 0 | 0 | 0 |
| Ryegrass, Italian | 0 | 0 | 90 | 80 | 30 | 0 | 0 | 0 | 70 | 0 | 0 | 0 |
| Soybean | 50 | 50 | 80 | 80 | 70 | 30 | 30 | 0 | 70 | 40 | 0 | 10 |
| Wheat | 0 | 0 | 20 | 10 | 10 | 0 | 0 | 0 | 10 | 0 | 0 | 0 |

| 31 g ai/ha | Compounds | | | | | | | | | | | | | |
|---|---|---|---|---|---|---|---|---|---|---|---|---|---|---|
| Postemergence | 1 | 2 | 3 | 4 | 10 | 11 | 12 | 13 | 14 | 15 | 16 | 17 | 18 | 19 |
| Barnyardgrass | 60 | 30 | 30 | 60 | 30 | 40 | 50 | 50 | 20 | 10 | 0 | 50 | 20 | 10 |
| Blackgrass | 40 | 20 | 0 | 30 | 40 | 50 | 20 | 50 | 10 | 0 | 0 | 40 | 10 | 0 |
| Corn | 20 | 10 | 0 | 30 | 30 | 10 | 10 | 20 | 10 | 10 | 0 | 20 | 0 | 10 |
| Foxtail, Green | 50 | 30 | 0 | 70 | — | — | — | — | — | — | — | — | 10 | 10 |
| Galium | 70 | 80 | 30 | 90 | 80 | 80 | 80 | 80 | 50 | 10 | 10 | 60 | 70 | 30 |
| Horseweed | 100 | 80 | 0 | 30 | 30 | 40 | 30 | 30 | 10 | 10 | 0 | 30 | 70 | 20 |
| Kochia | 90 | 90 | 20 | 90 | 90 | 90 | 90 | 90 | 80 | 50 | 30 | 90 | 60 | 90 |
| Oat, Wild | 30 | 10 | 0 | 10 | 20 | 20 | 20 | 20 | 10 | 0 | 0 | 10 | 10 | 0 |
| Pigweed, Palmer | 100 | 100 | 10 | 90 | 100 | 100 | 100 | 100 | 90 | 10 | 30 | 100 | 90 | 70 |
| Ragweed | 50 | 40 | 20 | 60 | 30 | 20 | 30 | 20 | 10 | 0 | 0 | 20 | 80 | 20 |
| Ryegrass, Italian | 50 | 30 | 0 | 60 | 10 | 50 | 40 | 30 | 10 | 0 | 0 | 20 | 0 | 0 |
| Soybean | 80 | 50 | 30 | 70 | 50 | 80 | 60 | 60 | 60 | 30 | 30 | 60 | 30 | 40 |
| Wheat | 10 | 10 | 0 | 0 | 0 | 10 | 10 | 10 | 0 | 0 | 0 | 10 | 0 | 0 |

| 31 g ai/ha | Compounds | | | | |
|---|---|---|---|---|---|
| Postemergence | 21 | 22 | 23 | 24 | 25 |
| Barnyardgrass | 0 | 0 | 0 | 50 | 0 |
| Blackgrass | 0 | 0 | 0 | 50 | 0 |
| Corn | 0 | 0 | 0 | 20 | 0 |
| Foxtail, Green | 0 | 0 | 0 | 30 | 0 |
| Galium | 0 | 70 | 0 | 70 | 0 |
| Horseweed | 0 | 0 | 0 | 20 | 0 |
| Kochia | 0 | 0 | 0 | 80 | 0 |
| Oat, Wild | 0 | 0 | 0 | 20 | 0 |
| Pigweed, Palmer | 0 | 0 | 0 | 70 | 10 |
| Ragweed | 0 | 0 | 20 | 20 | 0 |
| Ryegrass, Italian | 0 | 0 | 0 | 30 | 0 |
| Soybean | 30 | 0 | 0 | 30 | 20 |
| Wheat | 0 | 0 | 0 | 0 | 0 |

### Test B

Plant species in the flooded paddy test selected from barnyardgrass (Echinochloa crusgalli), ducksalad (Heteranthera limosa), rice (Oryza sativa), and sedge, umbrella (small-flower umbrella sedge, Cyperus difformis) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 10 to 14 d, after which time all species were compared to controls and visually evaluated. Plant response ratings, summarized in Table B, are based on a scale of 0 to 100 where 0 is no effect and 100 is complete control. A dash (-) response means no test result.

TABLE B

| 250 g ai/ha | Compounds | | | | | | | | | | | | | |
|---|---|---|---|---|---|---|---|---|---|---|---|---|---|---|
| Flood | 1 | 2 | 3 | 4 | 5 | 6 | 7 | 8 | 9 | 10 | 11 | 12 | 13 | 14 |
| Barnyardgrass | 35 | 20 | 0 | 45 | 0 | 0 | 35 | 0 | 10 | 50 | 50 | 70 | 65 | 30 |
| Ducksalad | 95 | 60 | 0 | 90 | 50 | 0 | 80 | 45 | 60 | 85 | 75 | 75 | 90 | 60 |
| Rice | 40 | 15 | 0 | 20 | 0 | 0 | 20 | 10 | 5 | 25 | 20 | 20 | 35 | 10 |
| Sedge, Umbrella | 95 | 98 | 0 | 95 | 45 | 0 | 80 | 25 | 55 | 95 | 95 | 95 | 98 | 95 |

| 250 g ai/ha | Compounds | | | | | | | | | | | |
|---|---|---|---|---|---|---|---|---|---|---|---|---|
| Flood | 15 | 16 | 17 | 18 | 19 | 21 | 22 | 23 | 24 | 25 | 26 | 27 |
| Barnyardgrass | 0 | 0 | 60 | 15 | 15 | 0 | 0 | 0 | 60 | 0 | 0 | 0 |
| Ducksalad | 0 | 0 | 75 | 70 | 85 | 0 | 0 | 0 | 30 | 0 | 0 | 0 |
| Rice | 0 | 0 | 15 | 40 | 15 | 0 | 0 | 0 | 65 | 0 | 0 | 0 |
| Sedge, Umbrella | 0 | 0 | 95 | 80 | 98 | 0 | 0 | 0 | 85 | 0 | 0 | 0 |

1. A compound of Formula 1, stereoisomers, N-oxides, and salts thereof, agricultural compositions containing them and their use as herbicides: 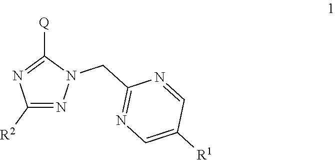
wherein
R¹ is halogen, cyano, nitro, C₁-C₄ alkyl, C₁-C₄ haloalkyl, C₃-C₇ cycloalkyl, C₂-C₅ alkenyl, C₂-C₅ alkynyl, C₁-C₄ alkylthio, C₁-C₄ alkylsulfinyl, C₁-C₄ alkylsulfonyl, C₁-C₄ alkoxy, C₂-C₅ alkylcarbonyl, C₂-C₅ alkoxycarbonyl, C₁-C₄ haloalkoxy or C₂-C₇ alkoxyalkyl;
R² is cyano, nitro, halogen, C₁-C₄ alkyl, C₁-C₄ haloalkyl, C₁-C₄ haloalkoxy, C₂-C₇ alkoxyalkyl, C₂-C₇ alkoxyalkoxy, C₃-C₇ cycloalkyl, C₂-C₅ alkylcarbonyl, C₂-C₅ alkoxycarbonyl, C₁-C₄ alkylthio, C₁-C₄ alkylsulfinyl, C₁-C₄ alkylsulfonyl, C₁-C₄ haloalkylthio, C₁-C₄ haloalkylsulfinyl or C₁-C₄ haloalkylsulfonyl;
Q is C₁-C₆ alkyl, C₁-C₆ haloalkyl, C₃-C₆ cycloalkyl, C₁-C₆ alkylthio, C₁-C₆ haloalkylthio, C₁-C₆ alkylsulfinyl, C₁-C₆ alkylsulfonyl, C₁-C₆ alkoxy, C₁-C₆ haloalkoxy, C₂-C₅ alkylcarbonyl, C₂-C₅ alkoxycarbonyl, C₂-C₅ haloalkylcarbonyl, C₂-C₅ haloalkoxycarbonyl or C₁-C₄ alkoxyalkyl; or G;
G is phenyl, or a 5- or 6-membered heterocyclic ring; the phenyl or heterocyclic ring unsubstituted or substituted with 1 or 2 R³; and
R³ is halogen, cyano, nitro, C₁-C₄ alkyl, C₁-C₄ haloalkyl, C₃-C₅ cycloalkyl, C₁-C₄ alkenyl, C₁-C₄ alkynyl, C₁-C₄ alkylthio, C₁-C₄ alkylsulfinyl, C₁-C₄ alkylsulfonyl, C₁-C₄ alkoxy, C₁-C₄ haloalkoxy, C₂-C₅ alkylcarbonyl, C₂-C₅ alkoxycarbonyl, C₁-C₄ alkoxyalkyl, C₁-C₄ alkylthio, C₁-C₄ alkylsulfinyl or C₁-C₄ alkylsulfonyl.

2. The compound of claim 1 wherein
R¹ is halogen, nitro or C₁-C₃ alkyl;
R² is cyano, nitro, halogen, C₁-C₄ alkyl or C₁-C₄ haloalkyl;
Q is C₁-C₆ alkyl, C₁-C₆ haloalkyl, C₁-C₆ alkylthio, C₁-C₆ haloalkylthio, C₁-C₆ alkylsulfinyl, C₁-C₆ alkylsulfonyl, C₁-C₆ alkoxy, C₁-C₆ haloalkoxy, C₂-C₅ alkylcarbonyl, C₂-C₅ alkoxycarbonyl, C₂-C₅ haloalkylcarbonyl, C₂-C₅ haloalkoxycarbonyl; or G;
G is phenyl substituted with 1 or 2 R³; or a 5- or 6-membered heterocyclic ring substituted with 1 or 2R³; and
R³ is halogen, cyano, C₁-C₄ alkyl, C₁-C₄ haloalkyl, C₁-C₄ alkylsulfinyl, C₁-C₄ alkylsulfonyl, C₁-C₄ alkoxy or C₁-C₄ alkylthio.

3. The compound of claim 2 wherein
R¹ is F, Cl, Br, nitro or CH₃;
R² is C₁-C₄ alkyl or C₁-C₄ haloalkyl;
Q is C₁-C₆ alkyl, C₁-C₆ haloalkyl, C₁-C₆ haloalkylthio, C₁-C₆ haloalkoxy, C₂-C₅ alkylcarbonyl, C₂-C₅ alkoxycarbonyl or C₂-C₅ haloalkylcarbonyl; or G;
G is selected from 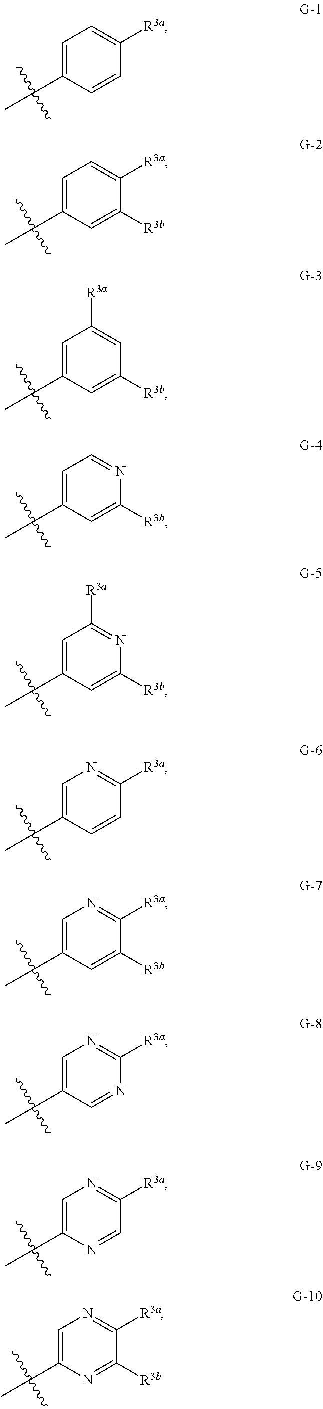 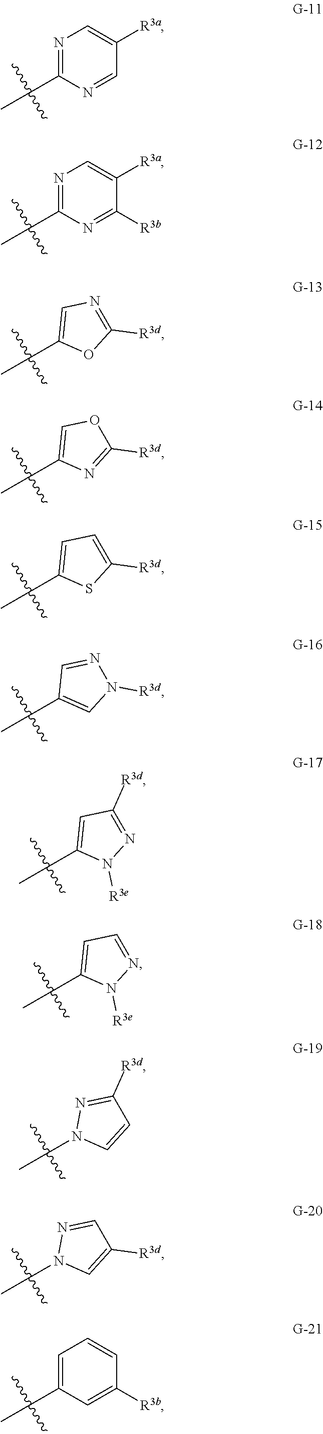 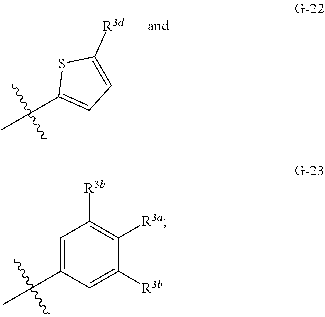
R³ is R^{3a}, R^{3b}, R^{3c}, R^{3d} or R^{3e}; and
R³ is halogen, C₁-C₃ alkyl or C₁-C₃ haloalkyl.

4. The compound of claim 3 wherein
R¹ is F, Cl, Br, nitro or CH₃;
R² is C₁-C₂ alkyl or C₁-C₂ haloalkyl;
Q is C₁-C₆ alkyl, C₃-C₆ haloalkyl, C₃-C₆ haloalkylthio, C₃-C₆ haloalkoxy, C₃-C₅ alkylcarbonyl, C₃-C₅ alkoxycarbonyl or C₃-C₅ haloalkylcarbonyl; or G;
G is selected from G-1, G-2, G-6, G-7, G-8 and G-15;
R^{3a} is Cl, F or CF₃;
R^{3b} is Cl or F; and
R^{3d} is Cl, CHF₂ or CF₃.

5. The compound of claim 4 wherein
R¹ is Cl, Br, nitro or CH₃;
R² is C₁-C₂ haloalkyl;
Q is C₁-C₆ alkyl or C₃-C₆ haloalkyl; or G;
G is G-1, G-2, G-6 and G-7; and
R^{3a} is Cl or CF₃.

6. The compound of claim 5 wherein
R¹ is Cl or Br;
R² is CHF₂, CF₃ or CF₂Cl;
Q is —CH₂CF₃, —CH₂CH₂CF₃—CH₂CH₂CH₂CF₃ or —OCH₂CH₂CH₂CF₃; or G;
G is G-1 and G-2.

7. The compound of claim 5 wherein
G is G-2;
R^{3a} is F; and
R^{3b} is Cl.

8. The compound of claim 5 wherein
G is G-2;
R^{3a} is F; and
R^{3b} is Cl.

9. A compound of claim 1 that is
5-chloro-2-[[3-(trifluoromethyl)-5-[4-(trifluoromethyl)phenyl]-1H-1,2,4-triazol-1-yl]methyl]pyrimidine.

10. A compound of Formula 6a 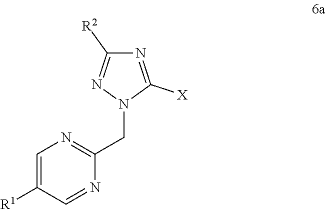
wherein
R¹ is halogen;
R² is C₁-C₄ haloalkyl; and
X is Br, Cl or I.

11. The compound of claim 10 wherein
R¹ is Cl;
R² is C₁-C₂ haloalkyl; and
X is Br or I.

12. The compound of claim 11 wherein
R² is CF₃; and
X is Br or I.

13. A herbicidal composition comprising a compound of claim 1 and at least one component selected from the group consisting of surfactants, solid diluents and liquid diluents.

14. The herbicidal composition of claim 13 and at least one additional active ingredient selected from the group consisting of other herbicides and herbicide safeners, and at least one component selected from the group consisting of surfactants, solid diluents and liquid diluents.

15. A herbicidal mixture comprising (a) a compound of claim 1, and (b) at least one additional active ingredient selected (b) at least one additional active ingredient selected from photosystem II inhibitors (b1), AHAS inhibitors (b2), ACCase inhibitors (b3), auxin mimics (b4), EPSP synthase inhibitors (b5), photosystem I electron diverters (b6), PPO inhibitors (b7), GS inhibitors (b8), VLCFA elongase inhibitors (b9), auxin transport inhibitors (b10), PDS inhibitors (b11), HPPD inhibitors (b12), DXP synthase inhibitors (b13), HST inhibitors (b14), cellulose biosynthesis inhibitors (b15), DHODH inhibitors (b16), other herbicides including mitotic disruptors, organic arsenicals, asulam, bromobutide, cinflubrolin, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb (b17), and herbicide safeners (b18); and salts of compounds of (b1) through (b18).

16. The herbicidal mixture of claim 15 wherein the at least one additional active ingredient is selected from DXP synthase inhibitors (b13), HST inhibitors (b14), cellulose biosynthesis inhibitors (b15), and DHODH inhibitors (b16); and salts thereof.

17. A method for controlling the growth of undesired vegetation comprising contacting the vegetation or its environment with a herbicidally effective amount of a compound of claim 1.

18. The method of claim 17 for controlling the growth of undesired vegetation comprising contacting the vegetation or its environment with a herbicidally effective amount of a composition of a compound of claim 1 and at least one additional herbicide or herbicide safener.
